(19) United States
(12) Patent Application Publication
(10) Pub. No.: US 2009/0317802 A1
(43) Pub. Date: Dec. 24, 2009

(54) Compositions and Methods to Monitor RNA Delivery to Cells

(76) Inventors: Sangeeta N. Bhatia, Lexington, MA (US); Austin M. Derfus, Solana Beach, CA (US); Alice A. Chen, Cambridge, MA (US)

Correspondence Address: CHOATE, HALL & STEWART LLP, TWO INTERNATIONAL PLACE, BOSTON, MA, 02110, US

(21) Appl. No.: 12/096,344

(22) PCT Filed: Dec. 8, 2006
(86) PCT No.: PCT/US06/46852

§ 371 Date: Feb. 18, 2009

Related U.S. Application Data
(60) Provisional application No. 60/749,376, filed on Dec. 9, 2005.

(51) Int. Cl. C12Q 1/68

(52) U.S. Cl. 435/6

(57) ABSTRACT

Methods and compositions for tracking or monitoring uptake of siRNA by mammalian cells are provided. The methods and compositions may be used to monitoring the silencing activity of the internalized siRNA. The compositions contain an siRNA, an optically or magnetically detectable nanoparticle such as a quantum dot and, optionally, a transfection reagent. Cells are contacted with an siRNA and an optically or magnetically detectable nanoparticle, optionally in the presence of a transfection reagent. Detection of internalized nanoparticles is indicative of siRNA uptake. The invention allows analysis, identification, processing, etc., of cells that have efficiently taken up siRNA. In one embodiment, cells are sorted into at least two populations based on the amount of siRNA taken up.

### GOVERNMENTAL SUPPORT

The United States Government has provided grant support utilized in the development of the present invention. In particular, National Institutes of Health contract number N01-C0-37117 has supported development of this invention. The United States Government may have certain rights in the invention.

### BACKGROUND OF THE INVENTION

Considerable attention has been devoted to developing reagents and methods for introducing nucleic acids into eukaryotic cells. Traditionally, most efforts have centered on the delivery of relatively large DNA constructs containing a gene of interest into the nucleus of eukaryotic cells in order to achieve either stable or transient increases in expression of the gene. More recently, with the discovery of RNA interference (RNAi), there has been increased interest in reagents and methods for delivering RNA to cells.

RNAi is a gene silencing mechanism triggered by double-stranded RNA (dsRNA) that has emerged as a powerful tool for studying gene function. Since the discovery of RNAi (1), the evolutionarily conserved process has been exploited to analyze the functions of nearly every gene in model organisms C. elegans (2, 3) and D. melanogaster (4) and a host of mammalian genes including approximately 23% of the sequenced human genes (5, 6). RNAi has also been used to effectively inhibit expression of viral genes in mammalian cells, resulting in inhibition of viral infection (45-47). In addition to viral target genes, RNAi has been used to silence expression of a wide range of endogenous disease-related genes in mammalian cells, suggesting a variety of potential therapeutic applications (see, e.g., 54).

RNAi is frequently achieved in mammalian cell culture or in vivo by the administration of short dsRNA duplexes, typically with symmetric 2-3 nucleotide 3′ overhangs, referred to as siRNA. If the RNAi effector sequence is potent and the siRNA delivered efficiently throughout the cell culture, remarkably specific post-transcriptional inhibition of gene expression can be achieved (7, 8). However, inefficient and heterogeneous delivery of siRNA is frequently observed in cell cultures, causing variable levels of gene silencing and potentially confounding the interpretation of genotype/phenotype correlations (9-12). Without the means to address and resolve transfection variability, the utility of RNAi in eukaryotes will only be fully realized in cell types that have been thoroughly optimized for siRNA delivery (13).

The importance of high transfection efficiency has been spotlighted by numerous reports investigating methods to either improve RNAi delivery (14-17) or screen for efficient knockdown. In the latter case, typical strategies involve monitoring fluorescently end-modified siRNAs (18, 19) or co-transfecting reporter plasmids and selecting for high transfection by fluorescence or antibiotic-resistance (20). These techniques enable one-time selection of highly transfected cells yet discard moderately-silenced cells, which may be of interest to the study. For example, varying degrees of RNAi-mediated downregulation in the tumor suppressor gene Trp53 have been shown to modulate expression of distinct pathological phenotypes both in vitro and in vivo (21). Moreover, rapid photobleaching of organic fluorophores and the limited selection of available reporters currently prevent RNAi tracking from being feasible in either long-term or multiplexed studies. The dyes commonly used to label siRNAs lose over half the intensity of fluorescent signal in 5-10 seconds (22, 23). Meanwhile, fluorescent reporter plasmids, although meant to be continuously expressed by the cells, can require as long as 2 hours after transcription for the functional protein to be observable (24). In addition, due to the limited availability of fluorophores and reporter proteins that have non-overlapping emission spectra, current screening methods that rely on exogenous administration of siRNAs to cells are incapable of simultaneous monitoring of multiple siRNA molecules.

Development of more effective methods for delivery of siRNA in vivo would enhance and expand the therapeutic possibilities of this technology. However, it has thus far been difficult to study siRNA delivery in animal models of human disease such as mice and rats. This difficulty confounds attempts to evaluate new siRNA delivery vehicles or to compare the efficacy and/or side effects of different siRNA sequences in vivo.

Thus there is a need in the art for improved methods for monitoring the delivery of functional RNAs such as siRNA to eukaryotic cells.

### SUMMARY OF THE INVENTION

The present invention provides compositions and methods for monitoring the delivery of RNA to cells. In one aspect, the invention provides an isolated composition comprising an optically or magnetically detectable nanoparticle and an RNAi agent. The nanoparticle may be physically associated with the RNAi agent. For example, in some embodiments of the invention, the RNAi agent and the nanoparticle are present in a complex with a transfection reagent. In some embodiments of the invention, the RNAi agent and the nanoparticle are either covalently or non-covalently conjugated to one another.

The invention further provides a composition comprising a nanoparticle, a functional RNA, and a transfection reagent. The functional RNA may be selected from the group consisting of: siRNAs, shRNAs, tRNAs, and ribozymes.

In one aspect, the invention provides a cell comprising an optically or magnetically detectable nanoparticle and a functional RNA, wherein the functional RNA was not synthesized by the cell.

In one aspect, the invention provides a kit comprising an optically or magnetically detectable nanoparticle and an RNAi agent. In certain embodiments of the invention, the RNAi agent is an siRNA and the nanoparticle is a quantum dot.

In one aspect, the invention provides a method of supplying an RNAi agent comprising steps of: (a) electronically receiving an order for an RNAi agent or an optically or magnetically detectable nanoparticle from a requestor; and (b) providing an RNAi agent and an optically or magnetically detectable nanoparticle to the requester, the nanoparticle being for use to track or monitor uptake of the RNAi agent by cells. In certain embodiments of the invention, the RNAi agent is an siRNA and the nanoparticle is a quantum dot.

In one aspect, the invention provides a method of monitoring delivery of a functional RNA to a cell comprising steps of: (a) contacting the cell with an optically or magnetically detectable nanoparticle and a functional RNA; and (b) analyzing the cell to detect the presence, absence, or amount of the nanoparticle in the cell, wherein presence of the nanoparticle in the cell is indicative of presence of the functional RNA in the cell. In certain embodiments of the invention, the functional RNA is a short RNAi agent (e.g., an siRNA), and the nanoparticle is a quantum dot. The amount of the nanoparticle in the cell is indicative of the amount and/or activity of the functional RNA in the cell in certain embodiments of the invention.

The invention further provides a method of monitoring gene silencing in a cell comprising steps of: (a) contacting the cell with an optically or magnetically detectable nanoparticle and an RNAi agent targeted to a gene; and (b) analyzing the cell to detect the presence, absence, or amount of the nanoparticle in the cell, wherein presence of the nanoparticle in the cell is indicative of silencing of the gene by the RNAi agent. The method may further comprise the step of separating the cells into at least two populations based on the amount of the nanoparticle in the cells.

The invention further provides a method of sorting cells comprising steps of: (a) contacting cells with an optically or magnetically detectable nanoparticle and a functional RNA; (b) analyzing the cells to detect the presence, absence, or amount of the nanoparticle in the cells; and (c) identifying the cells as belonging to one of at least two populations based on the presence, absence, or amount of the nanoparticle in the cells. The method may further comprise the step of physically separating the cells into at least two populations based on the presence, absence, or amount of the nanoparticle in the cells.

The invention further provides a method of sorting cells comprising steps of: (a) contacting cells with an optically or magnetically detectable nanoparticle and a functional RNA; (b) analyzing the cells to detect the presence, absence, or amount of the nanoparticle in the cells; and (c) physically separating the cells into at least two populations based on the presence, absence, or amount of the nanoparticle in the cells.

The invention further provides a method of preparing a composition comprising the step of: contacting an optically or magnetically detectable nanoparticle, a functional RNA, and a transfection reagent. The invention further provides a complex comprising an optically or magnetically detectable nanoparticle, a functional RNA, and a transfection reagent. In one embodiment, the nanoparticle is a quantum dot and the RNA is an siRNA.

The invention provides compositions and methods such as those described above comprising a multiplicity of different RNAs and a multiplicity of optically or magnetically distinguishable nanoparticles, wherein each of a multiplicity of different RNAs is physically associated with a nanoparticle that is distinguishable from nanoparticles associated with other RNAs. The invention may be used to track or monitor the uptake and/or activity of one RNA or of multiple RNAs in a eukaryotic cell in culture. Cells may be sorted, separated, and/or subject to further processing.

In various embodiments, the invention provides methods for the identification and/or selection of cells that have taken up siRNAs in an amount sufficient to silence one or more target genes, cells that have taken up approximately equal amounts of the same siRNA or of different siRNAs, cells that have taken up siRNAs in amounts that do not saturate the RNAi machinery, cells that have taken up siRNAs in amounts that do not result in non-sequence specific effects, cells that have taken up siRNAs in amounts that do not result in “off-target” silencing, etc.

In any of the compositions or methods of the invention, the RNA can be a short RNAi agent (e.g., an siRNA). In any of the compositions or methods of the invention the detectable nanoparticle can be a quantum dot. In any of the compositions or methods of the invention the nanoparticle may or may not have a biomolecule such as an endosome escape agent, a translocation peptide, or a nucleic acid attached thereto.

This application refers to various patent publications, all of which are incorporated herein by reference. For purposes of the present invention, the chemical elements are identified in accordance with the Periodic Table of the Elements, CAS version, Handbook of Chemistry and Physics, 75th Ed., inside cover, and specific functional groups are generally defined as described therein.

### BRIEF DESCRIPTION OF THE DRAWING

FIG. 1: Quantum dot/siRNA complexes allow sorting of gene silencing in cell populations. (Panel A) Schematic representation of cells co-transfected with quantum dots (QDs) and siRNA and analyzed for intracellular fluorescence by flow cytometry. Histograms depict fluorescence distributions of control murine fibroblast cells, Lmna siRNA-treated cells, and Lmna siRNA/QD-treated cells. FACS was used to gate and sort the high 10% (H) fluorescence and low 10% (L) fluorescence of each distribution. L⁻ and H⁻ point to gates for the siRNA only histogram. L⁺ and H⁺ indicate gates for the siRNA/QD histogram. (Panel B) Representative Western blot of Lamin A/C protein expression levels in sorted cells with β-actin as loading control. Control lanes are protein from cells mock-transfected with liposome reagent only and sorted (L, H). The absence of QDs is indicated by a minus sign (−) and the presence of QDs is indicate by a plus sign (+). (Panel C) Band densitometry analysis of Western blots from replicate experiments. Error bars represent standard error of the mean (n=3). ***P<0.001 (one-way ANOVA).

FIG. 2: Immunofluorescence staining of Lamin A/C nuclear protein. (Panel A) Unsorted cells (U) transfected with Lmna siRNA alone display heterogenous staining for Lamin A/C nuclear protein (red) throughout the cell population. White arrows highlight examples of cells with weak lamin staining among cells stained strongly for lamin. (Panel B) Cells co-transfected with Lmna siRNA and green QDs exhibit bright lamin staining and lack of QDs in low-gated (L⁺) cell subpopulations and (Panel C) weak lamin staining and presence of QDs in high-gated (H⁺) cell subpopulations (shown enlarged in inset). Scale bars 75 μm.

FIG. 3: Optimization of QD concentration for siRNA tracking. Lmna siRNA (100 nM) and 1, 2.5, 5, or 10 μg QD were co-transfected into murine fibroblasts and the cells FACS-sorted for the low 10% (L⁺) and high 10% (H⁺) of intracellular fluorescence distribution. (Panel A) Protein expression of sorted cells assayed by Western blot, β-actin loading control. Unsorted, lipofectamine only control (U) represented 100% lamin A/C protein expression. (Panel B) Western blot band densitometry analysis of L⁺ and H⁺ bands shows an optimum QD concentration for obtaining high-efficiency silencing. (Panel C) Band density difference (L⁺ minus H⁺) reveals an optimum QD concentration for sorting most efficiently silenced from least efficiently silenced subpopulations.

FIG. 4: Sorting the effects of double gene knockdowns using to colors of QDs. (Panel A) Schematic representation of cells transfected simultaneously with Lmna siRNA/green QD complexes and T-cad siRNA/orange QD complexes. The low 8% (L⁺⁺, where ++ designates the presence of two colors of QDs) and high 8% (H⁺⁺) of the dual fluorescence dot plot was gated and isolated using FACS. (Panel B) Representative Western blot and (Panel C) corresponding band densitometry analysis of lamin A/C and T-cadherin protein levels in control unsorted (U) cells, unsorted (U) T-cad siRNA-treated cells, sorted T-cad/QD-treated cells (L⁺, H⁺), and sorted dual siRNA/dual QD-treated (L⁺⁺, H⁺⁺) cells.

FIG. 5: Fluorescence/phase micrographs of two color QD transfections. (Panel A) Low-gated cells (L⁺⁺, where ++ indicates the presence of two colors of QD) nearly lack orange or green QDs. (Panel B) High-gated cells (H⁺⁺) fluoresce brightly with punctate green and orange QDs (enlarged in inset). Scale bars 100 μm.

FIG. 6: Significant downstream gene knockdown effects of T-cadherin gene silencing are observed only in a homogenously silenced cell population. Murine 3T3 fibroblasts transfected with T-cad siRNA alone or with T-cad siRNA/QD complexes were FACS-sorted for low 10% (L) or high 10% (H) intracellular fluorescence. Symbols − and + indicate the absence or presence of QD during transfection. To study the stabilizing effect of non-parenchymal cell (3T3 fibroblast) protein expression on liver-specific function, control or transfected/sorted 3T3 cells were added to hepatocyte cultures 24 hours after hepatocyte seeding. Liver-specific function was assayed by measuring albumin synthesis and cytochrome P450 1A1 (CYP1A1) activity of cultured media sampled at 72 and 96 hours after 3T3 seeding and averaged. Error bars represent standard error of the mean (n=3). *P<0.05, **P<0.01, ***P<0.001 (one-way ANOVA statistical analysis test).

FIG. 7: Knockdown efficacy is not improved by transfecting higher doses of siRNA. 3T3 murine fibroblasts were transfected with 100, 200, 300 or 400 nM Lmna siRNA and harvested for protein after 72 h. (Panel A) Representative Western blot of Lamin A/C protein levels, β-actin loading control. (Panel B) Band densitometry analysis from replicate experiments, where error bars represent standard error of the mean (n=2).

FIG. 8: QD-labeled and fluorescein-labeled siRNA fluorescence in 3T3 murine fibroblasts. After continuous mercury lamp exposure, QD fluorescence is shown in Panel A and siRNA fluorescence is shown in Panel B. Scale bars are 25 μm.

FIG. 9: Silencing activity of QD/siRNA conjugates in mammalian cells. The upper portion of the figure shows reagents used to synthesize the conjugates. The lower left portion of the figure shows silencing activity of siRNA or QD/siRNA conjugates in HeLa cells. The lower right portion of the figure shows signal obtained from the internalized QD/siRNA conjugates.

FIG. 10: Schematic diagram illustrating multifunctional nanoparticles for siRNA delivery.

FIG. 11: Uptake of unconjugated QDs or QDs conjugated with a variety of different moieties. A fluorescence histogram shows uptake by HeLa cells of unconjugated QDs or QDs conjugated with a variety of different moieties.

FIG. 12. Attachment of F3 peptide leads to QD internalization in HeLa cells. Thiolated peptides (F3 and KAREC control) and siRNA were conjugated to PEG-amino QD705 particles using sulfo-SMCC. Particles were filtered to remove excess peptide or siRNA, and incubated with HeLa cell monolayers for 4 hours. Flow cytometry indicated the F3 peptide is required for cell entry (Panel A). The addition of free P3 peptide inhibits F3-QD uptake, while KAREC peptide does not, suggesting the F3 peptide and F3-labeled particles target the same receptor (Panel B). In Panel C, the relationship between number of F3 peptides per QD and cell uptake was examined. In these experiments, FITC-labeled peptide was conjugated to QDs using sulfo-LC-SPDP. For each formulation (black circles), peptide:QD ratio was determined by measuring the QD concentration by absorbance, then treating the conjugate with 2-mercaptoethanol, filtering out the QDs, and measuring the FITC fluorescence. Cell uptake increases dramatically with peptide number, but appears to saturate around 10-15 F3s per QD.

FIG. 13. Conjugation of siRNA to QDs with cleavable or non-cleavable cross-linkers. Thiol-modified siRNA was attached to PEG-amino QDs using the water-soluble heterobifunctional cross-linkers sulfo-SMCC and sulfo-LC-SPDP (Panel A). The cross-link produced by SPDP is cleavable with 2-mercaptoethanol (2-ME), while the bond attained with SMCC is covalent. Gel electrophoresis of the disulfide-linked conjugates indicated that no siRNA are electrostatically bound to the conjugate (Panel B). Upon treatment with 2-ME, the QD/siRNA cross-link is reduced and the siRNA migrated down the gel alongside siRNA standards (Panel C). QD/siRNA conjugates (or siRNA alone) were delivered to EGFP-expressing HeLa cells using Lipofectamine 2000 (cationic liposome reagent). Cells were trypsinized and assayed by flow cytometry 48 h later. Comparison with control cells (treated with Lipofectamine alone) indicated the disulfide bond leads to superior EGFP knockdown (% reduction in geometric mean fluorescence) (Panel D). Comparing a dot-plot of cells treated with Lipofectamine alone (Panel E) or disulfide-linked QD/siRNA (Panel F) revealed a negative correlation between QD uptake and EGFP signal. Thus, the QD label can serve as a means of quantifying siRNA delivery and thus knockdown.

FIG. 14. Co-attachment of F3 peptide and siRNA cargo allows EGFP knockdown upon delivery and endosome escape. Due to a limited number of attachment sites on the QDs, the goal of co-attachment was to maximize siRNA loading while conjugating sufficient F3 peptides to allow internalization (>15). Varying the F3:siRNA ratio resulted in a number of formulations (black circles, Panel A), with superior QDs observed using a reaction ratio of 4:1 and resulting in ˜20 F3 peptide and ˜1 siRNA per QD. EGFP-expressing HeLa cells were treated with 50 mM F3/siRNA-QDs for four hours and then washed with cell media. When assayed for green fluorescence 48 hours later, no knockdown was observed (“control,” Panel B). When these cells were treated with cationic liposomes (Lipofectamine 2000) immediately after removing the QDs and washing, a ˜29% reduction in EGFP was observed. A lower concentration of QDs (10 nM) is less effective (21% knockdown). Incubation with KAREC-labeled particles followed by cationic liposomes leads to minimal particle internalization, and thus no knockdown. Fluorescence imaging of cells incubated with F3/siRNA QDs showed a reduced green fluorescence (Panel D), compared with control cells incubated with Lipofectamine alone (Panel C).

### DEFINITIONS

Approximately: As used herein, the terms “approximately” or “about” in reference to a number are generally taken to include numbers that fall within a range of 5% in either direction (greater than or less than) of the number unless otherwise stated or otherwise evident from the context (except where such number would exceed 100% of a possible value).

Conjugated: As used herein, the terms “conjugated,” “linked,” and “attached,” when used with respect to two or more moieties, means that the moieties are physically associated or connected with one another, either directly or via one or more additional moieties that serves as a linking agent, to form a structure that is sufficiently stable so that the moieties remain physically associated under the conditions in which structure is used, e.g., physiological conditions. Typically the moieties are attached either by one or more covalent bonds or by a mechanism that involves specific binding. Alternately, a sufficient number of weaker interactions can provide sufficient stability for moieties to remain physically associated.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism such as a non-human animal.

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically a reduction in the level of an mRNA results in a reduction in the level of a polypeptide translated therefrom. The level of expression may be determined using standard techniques for measuring mRNA or protein.

Isolated composition: As used herein, the term “isolated composition” refers to a composition present outside of a cell.

Isolated cell: As used herein, the term “isolated cell” refers to a cell not contained in a multi-cellular organism.

Liposomes: As used herein, the term “liposomes” refers to artificial microscopic spherical particles formed by a lipid-containing bilayer (or multilayers) enclosing an aqueous compartment.

RNAi: As used herein, the term “RNAi” refers to sequence specific inhibition of gene expression mediated by an at least partly double-stranded RNA molecule that contains a portion that is substantially complementary to a target gene (e.g., to an mRNA transcribed from the target gene). RNAi can occur via selective intracellular degradation of RNA and/or by translational repression.

RNAi agent: As used herein, the term “RNAi agent” refers to an at least partly double-stranded RNA molecule, optionally including one or more nucleotide analogs or modifications, having a structure characteristic of molecules that can mediate inhibition of gene expression through an RNAi mechanism. The RNAi agent includes a portion that is substantially complementary to a target gene.

Short RNAi agent: As used herein, the term “short RNAi agent” refers to an RNAi agent containing a dsRNA portion that is no greater than 50 base pairs in length, typically 30 base pairs or less in length, e.g., 17-29 base pairs in length. The term “short RNAi agent” includes siRNA and shRNA.

shRNA: As used herein, the term “shRNA” refers to an RNAi agent consisting of a single strand that contains substantially complementary portions capable of hybridizing to form a duplex structure sufficiently long to mediate RNAi (as described for siRNA duplexes), at least one single-stranded portion that forms a loop (typically from 4 to about 11 nucleotides in length) connecting adjacent termini of the duplex, and optionally an overhang. One of the portions that forms the duplex is substantially complementary to a portion of a target gene.

siRNA: As used herein, the term “siRNA” refers an RNAi agent containing a duplex portion formed from two independent strands, one of which is substantially complementary to a portion of a target gene over the portion that participates in duplex formation. Typically the duplex portion is about 17 to 29 base pairs in length, e.g., 19 base pairs in length. Typically one or both strands of the siRNA has a 2-3 nucleotide 3′ overhang.

Specific binding: As used herein, the term “specific binding” refers to non-covalent physical association of a first and a second moiety wherein the association between the first and second moieties is at least 100 times as strong as the association of either moiety with most or all other moieties present in the environment in which binding occurs. Binding of two or more entities may be considered specific if the equilibrium dissociation constant, K_{d}, is 10⁻⁶ M or less, 10⁻⁷ M or less, 10⁻⁸ M or less, or 10⁻⁹ M or less under the conditions employed, e.g., under physiological conditions such as those inside a cell or consistent with cell survival. Examples of specific binding interactions include antibody-antigen interactions, avidin-biotin interactions, hybridization between complementary nucleic acids, etc.

Subject: As used herein, the term “subject” refers to any multicellular organism to which a composition of this invention may be administered, e.g., for experimental, diagnostic, and/or therapeutic purposes. Typical subjects include animals, e.g., mammals such as mice, rats, rabbits, non-human primates, and humans.

Target gene: As used herein, the term “target gene” refers to any gene whose expression is inhibited by an RNAi agent.

Target transcript: As used herein, the term “target transcript” refers to any mRNA transcribed from a target gene.

Transfection reagent: As used herein, the term “transfection reagent” refers to any substance that enhances the transfer or uptake of an exogenous nucleic acid into a cell when the cell is contacted with the nucleic acid in the presence of the transfection reagent. In some embodiments, transfection reagents enhance the transfer of an exogenous nucleic acid, e.g., RNA, into mammalian cells.

Unnatural amino acid: As used herein, the term “unnatural amino acid” refers to any amino acid other than the 20 naturally-occurring amino acids found in naturally occurring proteins, and includes amino acid analogues. In general, any compound that can be incorporated into a polypeptide chain can be an unnatural amino acid. In some embodiments, such compounds have the chemical structure H₂N—CHR—CO₂H. The alpha-carbon may be in the L-configuration, as in naturally occurring amino acids, or may be in the D-configuration.

### DETAILED DESCRIPTION OF CERTAIN EMBODIMENTS OF THE INVENTION

### I. Overview

The present invention provides compositions and methods for monitoring the uptake of RNA by eukaryotic cells. A variety of different classes of RNA molecules can be monitored. For example, the RNA may be a short RNAi agent such as an siRNA that inhibits gene expression or may be a transfer RNA (tRNA) that functions in protein synthesis. In certain embodiments of the invention, the amount of RNA delivered to the interior of a cell serves as an indicator of the activity of the RNA in the cell. For example, in certain embodiments of the invention, RNA uptake, as determined in accordance with the invention, correlates with the activity of the RNA in the cell. The invention thus provides means for tracking, monitoring, and/or measuring the activity of an RNA in a eukaryotic cell.

The methods of the invention involve contacting a cell or, more typically, a plurality of cells, with an RNA and a detectable nanoparticle, e.g., an optically or magnetically detectable nanoparticle. The nanoparticle has dimensions small enough to allow it to enter the cell. Both the nanoparticle and the RNA are taken up by the cell, i.e., they are delivered to the interior of the cell. Delivery can be achieved in any of a number of ways as discussed further below. Following delivery of the RNA and the nanoparticle to the interior of the cell, the cell is analyzed to detect the nanoparticle. The presence of the nanoparticle in the cell serves as an indicator of the presence of the RNA in the cell. Optionally the cell is sorted based on a property of the nanoparticle, e.g., an optical or magnetic property. Thus detecting the nanoparticle allows identification, isolation, selection, or sorting of cells that have taken up the RNA.

In typical embodiments of the invention, the cell or plurality of cells is contacted with a plurality of nanoparticles comprising or consisting of nanoparticles that have one or more substantially uniform optical and/or magnetic properties. Thus the term “nanoparticle” as used herein can refer to either a single nanoparticle or to a population of nanoparticles comprising or consisting of nanoparticles having one or more substantially uniform optical and/or magnetic properties. The optical and/or magnetic properties of the nanoparticles that make up a population need not be identical but need only be sufficiently similar so that the nanoparticles can be effectively detected and can be distinguished from other populations of nanoparticles, e.g., in embodiments of the invention in which the cell(s) are contacted with more than one population of nanoparticles. Typically the particles of a population having substantially uniform optical or magnetic properties will be substantially similar in size, shape, and/or composition. When cells are contacted with a population of nanoparticles, the magnitude of the signal acquired from a particular cell is, on the average, indicative of the number of nanoparticles taken up by the cell. Suitable nanoparticles include, e.g., quantum dots (QDs), fluorescent or luminescent nanoparticles, and magnetic nanoparticles. Any optical or magnetic property or characteristic of the nanoparticle(s) can be detected.

In certain embodiments of the invention, the number of nanoparticles taken up by the cell is positively correlated with the amount of RNA taken up by the cell, i.e., with the number of RNA molecules taken up by the cell. In other words, if the number of nanoparticles present in two cells is compared, the cell that contains a larger number of nanoparticles typically contains a larger amount of RNA. The correlation between nanoparticle and RNA uptake can be linear or non-linear and can exist over all or part of a range of nanoparticle and/or RNA concentrations to which a cell is exposed. In certain embodiments of the invention, the nanoparticle and the RNA are physically associated, so that they are taken up together. For example, the nanoparticle and the RNA may be associated in a complex with a transfection reagent. In certain embodiments of the invention, the transfection reagent both enhances uptake of the nanoparticle and the RNA by the cell and serves to physically associate the nanoparticle and the RNA with one another.

As described in Examples 1 and 2, using the inventive QD/siRNA co-delivery technique, cellular fluorescence was shown to correlate with level of silencing, allowing collection of a uniformly silenced cell population by fluorescence-activated cell sorting (FACS). Importantly, the present invention demonstrates that the presence of optically detectable nanoparticles such as QDs within mammalian cells does not interfere with RNAi even when the particles are present in large numbers. The superior brightness and photostability of QD probes in cells sustained not only FACS, but also live imaging and immunostaining procedures. As described in Example 3, with the use of two QD colors and two siRNAs, the method was used to generate cell populations with multiplexed levels of knockdown. Example 4 shows that a homogenously silenced cell population generated using this method is essential to observing the phenotypic effects of decreased T-cadherin protein expression on cell-cell communication between hepatocytes and non-parenchymal cells, thus providing a sample of the wide range of biologically relevant discoveries that are made possible by the methods of the invention.

The following section describes a variety of RNA molecules whose uptake by and/or activity in eukaryotic cells can be monitored according to the invention. Subsequent sections describe nanoparticles and their detection, transfection reagents, cells, and other components of the invention.

### II. Functional RNAs and their Activities

The invention can be used to monitor RNA molecules of a wide variety of types within cells. In certain embodiments of the invention, the RNA is an RNA that does not code for a protein but instead belongs to a class of RNA molecules whose members characteristically possess one or more different functions or activities within a cell. Such RNAs are referred to herein as “functional RNAs.” It will be appreciated that the relative activities of functional RNA molecules having different sequences may differ and may depend at least in part on the particular cell type in which the RNA is present. Thus the term “functional RNA” is used herein to refer to a class of RNA molecule and is not intended to imply that all members of the class will in fact display the activity characteristic of that class under any particular set of conditions. While the scope of RNAs whose cellular uptake and/or activity can be monitored and tracked is in no way limited, the invention finds particular use for tracking and monitoring the uptake and/or activity of short RNAi agents and tRNAs.

As mentioned above, RNAi is an evolutionarily conserved process in which presence of an at least partly double-stranded RNA molecule in a eukaryotic cell leads to sequence-specific inhibition of gene expression. RNAi was originally described as a phenomenon in which the introduction of long dsRNA (typically hundreds of nucleotides) into a cell results in degradation of mRNA containing a region complementary to one strand of the dsRNA (U.S. Pat. No. 6,506,559 and ref. 1). Subsequent studies in Drosophila showed that long dsRNAs are processed by an intracellular RNase III-like enzyme called Dicer into smaller dsRNAs primarily comprised of two ˜21 nucleotide (nt) strands that form a 19 base pair duplex with 2 nt 3′ overhangs at each end and 5′-phosphate and 3′-hydroxyl groups (see, e.g., PCT Pub. No. WO 01/75164; U.S. Pub. Nos. 20020086356 and 20030108923; and refs. 49-50).

Short dsRNAs having structures such as this, referred to as siRNAs, silence expression of genes that include a region that is substantially complementary to one of the two strands. This strand is referred to as the “antisense” or “guide” strand, with the other strand often being referred to as the “sense” strand. The siRNA is incorporated into a ribonucleoprotein complex termed the RNA-induced silencing complex (RISC) that contains member(s) of the Argonaute protein family. Following association of the siRNA with RISC, a helicase activity unwinds the duplex, allowing an alternative duplex to form the guide strand and a target mRNA containing a portion substantially complementary to the guide strand. An endonuclease activity associated with the Argonaute protein(s) present in RISC is responsible for “slicing” the target mRNA, which is then further degraded by cellular machinery.

Considerable progress towards the practical application of RNAi was achieved with the discovery that exogenous introduction of siRNAs into mammalian cells can effectively reduce the expression of target genes in a sequence-specific manner via the mechanism described above. A typical siRNA structure includes a 19 nucleotide double-stranded portion, comprising a guide strand and an antisense strand. Each strand has a 2 nt 3′ overhang. Typically the guide strand of the siRNA is perfectly complementary to its target gene and mRNA transcript over at least 17-19 contiguous nucleotides, and typically the two strands of the siRNA are perfectly complementary to each other over the duplex portion. However, as will be appreciated by one of ordinary skill in the art, perfect complementarity is not required. Instead, one or more mismatches in the duplex formed by the guide strand and the target mRNA is often tolerated, particularly at certain positions, without reducing the silencing activity below useful levels. For example, there may be 1, 2, 3, or even more mismatches between the target mRNA and the guide strand (disregarding the overhangs). Thus, as used herein, two nucleic acid portions such as a guide strand (disregarding overhangs) and a portion of a target mRNA that are “substantially complementary” may be perfectly complementary (i.e., they hybridize to one another to form a duplex in which each nucleotide is a member of a complementary base pair) or they may have a lesser degree of complementarity sufficient for hybridization to occur. One of ordinary skill in the art will appreciate that the two strands of the siRNA duplex need not be perfectly complementary. Typically at least 80%, preferably at least 90%, or more of the nucleotides in the guide strand of an effective siRNA are complementary to the target mRNA over at least about 19 contiguous nucleotides. The effect of mismatches on silencing efficacy and the locations at which mismatches may most readily be tolerated are areas of active study (see, e.g., 53).

It will be appreciated that molecules having the appropriate structure and degree of complementarity to a target gene will exhibit a range of different silencing efficiencies. A variety of additional design criteria have been developed to assist in the selection of effective siRNA sequences. Numerous software programs that can be used to choose siRNA sequences that are predicted to be particularly effective to silence a target gene of choice are available (see, e.g., 51-52).

As will be appreciated by one of ordinary skill in the art, RNAi may be effectively mediated by RNA molecules having a variety of structures that differ in one or more respects from that described above. For example, the length of the duplex can be varied (e.g., from about 17-29 nucleotides); the overhangs need not be present and, if present, their length and the identity of the nucleotides in the overhangs can vary (though most commonly symmetric dTdT overhangs are employed in synthetic siRNAs).

Additional structures, referred to as short hairpin RNAs (shRNAs), are capable of mediating RNA interference. An shRNA is a single RNA strand that contains two complementary regions that hybridize to one another to form a double-stranded “stem,” with the two complementary regions being connected by a single-stranded loop. shRNAs are processed intracellularly by Dicer to form an siRNA structure containing a guide strand and an antisense strand. While shRNAs can be delivered exogenously to cells, more typically intracellular synthesis of shRNA is achieved by introducing a plasmid or vector containing a promoter operably linked to a template for transcription of the shRNA into the cell, e.g., to create a stable cell line or transgenic organism.

While sequence-specific cleavage of target mRNA is currently the most widely used means of achieving gene silencing by exogenous delivery of short RNAi agents to cells, additional mechanisms of sequence-specific silencing mediated by short RNA species are known. For example, post-transcriptional gene silencing mediated by small RNA molecules can occur by mechanisms involving translational repression. Certain endogenously expressed RNA molecules form hairpin structures containing an imperfect duplex portion in which the duplex is interrupted by one or more mismatches and/or bulges. These hairpin structures are processed intracellularly to yield single-stranded RNA species referred to as known as microRNAs (miRNAs), which mediate translational repression of a target transcript to which they hybridize with less than perfect complementarity. siRNA-like molecules designed to mimic the structure of miRNA precursors have been shown to result in translational repression of target genes when administered to mammalian cells.

Thus the exact mechanism by which a short RNAi agent inhibits gene expression appears to depend, at least in part, on the structure of the duplex portion of the RNAi agent and/or the structure of the hybrid formed by one strand of the RNAi agent and a target transcript. RNAi mechanisms and the structure of various RNA molecules known to mediate RNAi, e.g., siRNA, shRNA, miRNA and their precursors, have been extensively reviewed (see, e.g., 54-56). It is to be expected that future developments will reveal additional mechanisms by which RNAi may be achieved and will reveal additional effective short RNAi agents. Any currently known or subsequently discovered short RNAi agents are within the scope of the present invention.

A short RNAi agent that is tracked and/or monitored according to the methods of the invention and/or is present in a composition of the invention may be designed to silence any eukaryotic gene. The gene can be a mammalian gene, e.g., a human gene. The gene can be a wild type gene, a mutant gene, an allele of a polymorphic gene, etc. The gene can be disease-associated, e.g., a gene whose over-expression, under-expression, or mutation is associated with or contributes to development or progression of a disease. For example, the gene can be oncogene. The gene can encode a receptor or putative receptor for an infectious agent such as a virus (see, e.g., ref. 54 for specific examples).

In some embodiments, tRNAs are functional RNA molecules whose delivery to eukaryotic cells can be monitored using the compositions and methods of the invention. The structure and role of tRNAs in protein synthesis is well known (Soll and Rajbhandary, (eds.) tRNA: Structure, Biosynthesis, and Function, ASM Press, 1995). The cloverleaf shape of tRNAs includes several double-stranded “stems” that arise as a result of formation of intramolecular base pairs between complementary regions of the single tRNA strand. There is considerable interest in the synthesis of polypeptides that incorporate unnatural amino acids such as amino acid analogs or labeled amino acids at particular positions within the polypeptide chain (see, e.g., Köhrer and RajBhandary, “Proteins carrying one or more unnatural amino acids,” Chapter 33, In Ibba et al., (eds.), Aminoacyl-tRNA Synthetases, Landes Bioscience, 2004). One approach to synthesizing such polypeptides is to deliver a suppressor tRNA that is aminoacylated with an unnatural amino acid to a cell that expresses an mRNA that encodes the desired polypeptide but includes a nonsense codon at one or more positions. The nonsense codon is recognized by the suppressor tRNA, resulting in incorporation of the unnatural amino acid into a polypeptide encoded by the mRNA (48, 57). However, as in the case of siRNA delivery, existing methods of delivering tRNAs to cells result in variable levels of delivery, complicating efforts to analyze such proteins and their effects on cells.

The invention contemplates the delivery of tRNAs, e.g., suppressor tRNAs, and optically or magnetically detectable nanoparticles to eukaryotic cells in order to track and monitor tRNA uptake and/or to track and monitor the synthesis of proteins that incorporate an unnatural amino acid with which the tRNA is aminoacylated. The analysis of proteins that incorporate one or more unnatural amino acids has a wide variety of applications. For example, incorporation of amino acids modified with detectable (e.g., fluorescent) moieties can allow the study of protein trafficking, secretion, etc., with minimal disturbance to the native protein structure. Alternatively or additionally, incorporation of reactive moieties (e.g., photoactivatable and/or cross-linkable groups) can be used to identify protein interaction partners and/or to define three-dimensional structural motifs. Incorporation of phosphorylated amino acids such as phosphotyrosine, phosphothreonine, or phosphoserine, or analogs thereof, into proteins can be used to study cell signaling pathways and requirements.

In one embodiment of the invention, the functional RNA is a ribozyme.

RNAs such as RNAi agents, tRNAs, ribozymes, etc., for delivery to eukaryotic cells may be prepared according to any available technique including, but not limited to chemical synthesis, enzymatic synthesis, enzymatic or chemical cleavage of a longer precursor, etc. Methods of synthesizing RNA molecules are known in the art (see, e.g., Gait, M. J. (ed.) Oligonucleotide synthesis: a practical approach, Oxford [Oxfordshire], Washington, D.C.: IRL Press, 1984; and Herdewijn, P. (ed.) Oligonucleotide synthesis: methods and applications, Methods in molecular biology, v. 288 (Clifton, N.J.) Totowa, N.J.: Humana Press, 2005). Short RNAi agents such as siRNAs are commercially available from a number of different suppliers. Pre-tested siRNAs targeted to a wide variety of different genes are available, e.g., from Ambion (Austin, Tex.), Dharmacon (Lafayette, Colo.), Sigma-Aldrich (St. Louis, Mo.).

When siRNAs are synthesized in vitro the two strands are typically allowed to hybridize before contacting them with cells. It will be appreciated that the resulting siRNA composition need not consist entirely of double-stranded (hybridized) molecules. For example, an RNAi agent commonly includes a small proportion of single-stranded RNA. Generally, at least approximately 50%, at least approximately 90%, at least approximately 95%, or even at least approximately 99-100% of the RNAs in an siRNA composition are double-stranded when contacted with cells. However, a composition containing a lower proportion of dsRNA may be used, provided that it contains sufficient dsRNA to be effective.

It will be appreciated by those of ordinary skill in the art that synthetic RNAs such as RNAi agents may comprise nucleotides entirely of the types found in naturally occurring nucleic acids, or may instead include one or more nucleotide analogs or have a structure that otherwise differs from that of a naturally occurring nucleic acid. U.S. Pat. Nos. 6,403,779; 6,399,754; 6,225,460; 6,127,533; 6,031,086; 6,005,087; 5,977,089; and references therein disclose a wide variety of specific nucleotide analogs and modifications that may be used in a functional RNA. See Crooke, S. (ed.) Antisense Drug Technology: Principles, Strategies, and Applications (1^{st} ed), Marcel Dekker; ISBN: 0824705661; 1st edition (2001) and references therein. For example, 2′-modifications include halo, alkoxy and allyloxy groups. In some embodiments, the 2′-OH group is replaced by a group selected from H, OR, R, halo, SH, SR₁, NH₂, NH_{R}, NR₂ or CN, wherein R is C₁-C₆ alkyl, alkenyl or alkynyl and halo is F, Cl, Br or I. Examples of modified linkages include phosphorothioate and 5′-N-phosphoramidite linkages.

Nucleic acids containing a variety of different nucleotide analogs, modified backbones, or non-naturally occurring internucleoside linkages can effectively mediate RNAi provided that they have contain a guide strand with a nucleobase sequence that is sufficiently complementary to the target gene. In some cases, RNAi agents containing such modifications display improved properties relative to nucleic acids consisting only of naturally occurring nucleotides. For example, the structure of an siRNA may be stabilized by including nucleotide analogs at the 3′ end of one or both strands order to reduce digestion, e.g., by exonucleases.

Modified nucleic acids need not be uniformly modified along the entire length of the molecule. Different nucleotide modifications and/or backbone structures may exist at various positions in the nucleic acid. One of ordinary skill in the art will appreciate that the nucleotide analogs or other modification(s) may be located at any position(s) of an RNAi agent such that the target-specific silencing activity is not substantially affected. The modified region may be at the 5′-end and/or the 3′-end of one or both strands. For example, modified siRNAs in which ˜1-5 residues at the 5′ and/or 3′ end of either of both strands are nucleotide analogs and/or have a backbone modification have been employed. The modification may be a 5′ or 3′ terminal modification. One or both nucleic acid strands of an active RNAi agent may comprise at least 50% unmodified RNA, at least 80% modified RNA, at least 90% unmodified RNA, or 100% unmodified RNA. In certain embodiments of the invention, one or more of the nucleic acids in an RNAi agent comprises 100% unmodified RNA within the portion of the guide strand that participates in duplex formation with a target nucleic acid.

RNAi agents may, for example, contain a modification to a sugar, nucleoside, or internucleoside linkage such as those described in U.S. Pub. Nos. 2003/0175950, 2004/0192626, 2004/0092470, 2005/0020525, and 2005/0032733. Studies describing the effect of a variety of different siRNA modifications have been reviewed (see ref. 18). The present invention encompasses the use of an RNAi agent having any one or more of the modification described therein. For example, a number of terminal conjugates, e.g., lipids such as cholesterol, lithocholic acid, aluric acid, or long alkyl branched chains have been reported to improve cellular uptake. Analogs and modifications may be tested using, e.g., using assays such as Western blots, immunofluorescence, or any appropriate assay known in the art, in order to select those that effectively reduce expression of target genes and/or result in improved stability, uptake, etc.

### III. Nanoparticles and Detection Methods

A variety of different nanoparticles are of use in the invention. In general, the nanoparticles have detectable optical and/or magnetic properties, though nanoparticles that may be detected by other approaches could be used. An optically detectable nanoparticle is one that can be detected within a living cell using optical means compatible with cell viability. Optical detection is accomplished by detecting the scattering, emission, and/or absorption of light that falls within the optical region of the spectrum, i.e., that portion of the spectrum extending from approximately 180 nm to several microns. Optionally a sample containing cells is exposed to a source of electromagnetic energy. In some embodiments of the invention, absorption of electromagnetic energy (e.g., light of a given wavelength) by the nanoparticle or a component thereof is followed by the emission of light at longer wavelengths, and the emitted light is detected. In some embodiments, scattering of light by the nanoparticles is detected. In certain embodiments of the invention, light falling within the visible portion of the electromagnetic spectrum, i.e., the portion of the spectrum that is detectable by the human eye (approximately 400 nm to approximately 700 nm) is detected. In some embodiments of the invention, light that falls within the infrared or ultraviolet region of the spectrum is detected.

The optical property can be a feature of an absorption, emission, or scattering spectrum or a change in a feature of an absorption, emission, or scattering spectrum. The optical property can be a visually detectable feature such as, for example, color, apparent size, or visibility (i.e. simply whether or not the particle is visible under particular conditions). Features of a spectrum include, for example, peak wavelength or frequency (wavelength or frequency at which maximum emission, scattering intensity, extinction, absorption, etc. occurs), peak magnitude (e.g., peak emission value, peak scattering intensity, peak absorbance value, etc.), peak width at half height, or metrics derived from any of the foregoing such as ratio of peak magnitude to peak width. Certain spectra may contain multiple peaks, of which one is typically the major peak and has significantly greater intensity than the others. Each spectral peak has associated features. Typically, for any particular spectrum, spectral features such as peak wavelength or frequency, peak magnitude, peak width at half height, etc., are determined with reference to the major peak. The features of each peak, number of peaks, separation between peaks, etc., can be considered to be features of the spectrum as a whole. The foregoing features can be measured as a function of the direction of polarization of light illuminating the particles; thus polarization dependence can be measured. Features associated with hyper-Rayleigh scattering can be measured. Fluorescence detection can include detection of fluorescence modes.

Intrinsically fluorescent or luminescent nanoparticles, nanoparticles that comprise fluorescent or luminescent moieties, plasmon resonant nanoparticles, and magnetic nanoparticles are among the detectable nanoparticles that are used in various embodiments of the invention. Such particles can have a variety of different shapes including spheres, oblate spheroids, cylinders, shells, cubes, pyramids, rods (e.g., cylinders or elongated structures having a square or rectangular cross-section), tetrapods (particles having four leg-like appendages), triangles, prisms, etc. In general, the nanoparticles should have dimensions small enough to allow their uptake by eukaryotic cells. Typically the nanoparticles have a longest straight dimension (e.g., diameter) of 200 nm or less. In some embodiments, the nanoparticles have a diameter of 100 nm or less. Smaller nanoparticles, e.g., having diameters of 50 nm or less, e.g., 5-30 nm, are used in some embodiments of the invention. In some embodiments, the term “nanoparticle” encompasses atomic clusters, which have a typical diameter of 1 nm or less and generally contain from several (e.g., 3-4) up to several hundred atoms.

The nanoparticles can be solid or hollow and can comprise one or more layers (e.g., nanoshells, nanorings). They may have a core/shell structure, wherein the core(s) and shell(s) can be made of different materials. In certain embodiments of the invention, they are composed of either gradient or homogeneous alloys. In certain embodiments of the invention, the nanoparticles are composite particles made of two or more materials, of which one, more than one, or all of the materials possesses an optically or magnetically detectable property.

It is often desirable to use a population of nanoparticles that is relatively uniform in terms of size, shape, and/or composition so that each particle has similar properties, e.g., similar optical or magnetic properties. For example, at least 80%, at least 90%, or at least 95% of the particles may have a diameter or longest straight line dimension that falls within 5%, 10%, or 20% of the average diameter or longest straight line dimension.

In certain embodiments of the invention, one or more substantially uniform populations of particles is used, e.g., 2, 3, 4, 5, or more substantially uniform populations having distinguishable optical and/or magnetic properties. Each population of particles is associated with an RNA. Use of multiple distinguishable particle populations allows tracking of multiple different RNA species concurrently. It will be appreciated that a combination of two or more populations having distinguishable properties can be considered to be a single population. It will further be appreciated that combining two or more populations of particles in different ratios can expand the range of coding possibilities (see, e.g., (26). The present invention encompasses any suitable means of relating the identity of an RNA to a population of nanoparticles such that detecting the nanoparticles in a cell is indicative of the presence of the RNA in a cell.

Nanoparticles comprising one or more optically or magnetically detectable materials may have a coating layer. Use of a biocompatible coating layer can be advantageous, e.g., if the particles contain materials that are toxic to cells. Suitable coating materials include, but are not limited to, proteins such as bovine serum albumin (BSA), polyethylene glycol (PEG) or a PEG derivative, phospholipid-(PEG), silica, lipids, carbohydrates such as dextran, etc. Coatings may be applied or assembled in a variety of ways such as by dipping, using a layer-by-layer technique, by self-assembly, etc. Self-assembly refers to a process of spontaneous assembly of a higher order structure that relies on the natural attraction of the components of the higher order structure (e.g., molecules) for each other. It typically occurs through random movements of the molecules and formation of bonds based on size, shape, composition or chemical properties.

In certain embodiments of the invention, the nanoparticles are quantum dots (QDs). QDs are bright, fluorescent nanocrystals with physical dimensions small enough such that the effect of quantum confinement gives rise to unique optical and electronic properties. Semiconductor QDs are often composed of atoms from groups II-VI or III-V in the periodic table, but other compositions are possible (see, e.g., ref. 58, describing gold QDs). By varying their size and composition, the emission wavelength can be tuned (i.e., adjusted in a predictable and controllable manner) from the blue to the near infrared. QDs generally have a broad absorption spectrum and a narrow emission spectrum. Thus different QDs having distinguishable optical properties (e.g., peak emission wavelength) can be excited using a single source. QDs are brighter than most conventional fluorescent dyes by approximately 10-fold (22, 27) and have been significantly easier to detect than GFP among background autofluorescence in vivo (27). Furthermore, QDs are far less susceptible to photobleaching, fluorescing more than 20 times longer than conventional fluorescent dyes under continuous mercury lamp exposure (28).

QDs and methods for their synthesis are well known in the art (see, e.g., U.S. Pat. Nos. 6,322,901; 6,576,291; and 6,815,064). QDs can be rendered water soluble by applying coating layers comprising a variety of different materials (see, e.g., U.S. Pat. Nos. 6,423,551; 6,251,303; 6,319,426; 6,426,513; 6,444,143; and 6,649,138). For example, QDs can be solubilized using amphiphilic polymers. Exemplary polymers that have been employed include octylamine-modified low molecular weight polyacrylic acid, polyethylene-glycol (PEG)-derivatized phospholipids, polyanhydrides, block copolymers, etc. (27). QDs can be conjugated with a variety of different biomolecules such as nucleic acids, polypeptides, antibodies, streptavidin, lectins, and polysaccharides, e.g., via any of a number of different functional groups or linking agents that can be directly or indirectly linked to a coating layer (see, e.g., U.S. Pat. Nos. 5,990,479; 6,207,392; 6,251,303; 6,306,610; 6,325,144; and 6,423,551).

The inventors and others have shown that QDs can be rendered non-cytotoxic (25) and innocuous to normal cell physiology and common cellular assays, such as immunostaining and reporter gene expression (26). For example, QDs can be coated with PEG as described in Example 1 (see ref. 28). In one embodiment, QDs are encapsulated with a high molecular weight ABC triblock copolymer (27). Features and uses of QDs, optionally modified with affinity agents such as antibodies, have been reviewed (see, e.g., ref. 59-60). QDs with a wide variety of absorption and emission spectra are commercially available, e.g., from Quantum Dot Corp. (Hayward Calif.; now owned by Invitrogen) or from Evident Technologies (Troy, N.Y.). For example, QDs having peak emission wavelengths of approximately 525, 535, 545, 565, 585, 605, 655, 705, and 800 nm are available. Thus the QDs can have a range of different colors across the visible portion of the spectrum and in some cases even beyond.

Fluorescence or luminescence can be detected using any approach known in the art including, but not limited to, spectrometry, fluorescence microscopy, flow cytometry, etc. Spectrofluorometers and microplate readers are typically used to measure average properties of a sample while fluorescence microscopes resolve fluorescence as a function of spatial coordinates in two or three dimensions for microscopic objects (e.g., less than ˜0.1 mm diameter). Microscope-based systems are thus suitable for detecting and optionally quantitating nanoparticles inside individual cells.

Flow cytometry measures properties such as light scattering and/or fluorescence on individual cells in a flowing stream, allowing subpopulations within a sample to be identified, analyzed, and optionally quantitated (see, e.g., Mattheakis et al., 2004, Analytical Biochemistry, 327:200). Multiparameter flow cytometers are available. In certain embodiments of the invention, laser scanning cytometery is used (77). Laser scanning cytometry can provide equivalent data to a flow cytometer but is typically applied to cells on a solid support such as a slide. It allows light scatter and fluorescence measurements and records the position of each measurement. Cells of interest may be re-located, visualized, stained, analyzed, and/or photographed. Laser scanning cytometers are available, e.g., from CompuCyte (Cambridge, Mass.).

In certain embodiments of the invention, an imaging system comprising an epifluorescence microscope equipped with a laser (e.g., a 488 nm argon laser) for excitation and appropriate emission filter(s) is used. The filters should allow discrimination between different populations of nanoparticles used in the particular assay. For example, in one embodiment, the microscope is equipped with fifteen 10 nm bandpass filters spaced to cover portion of the spectrum between 520 and 660 nm, which would allow the detection of a wide variety of different fluorescent particles. Fluorescence spectra can be obtained from populations of nanoparticles using a standard UV/visible spectrometer.

In certain embodiments of the invention, the optically detectable nanoparticles are metal nanoparticles. Metals of use in the nanoparticles include, but are not limited to, gold, silver, iron, cobalt, zinc, cadmium, nickel, gadolinium, chromium, copper, manganese, palladium, tin, and alloys thereof. Oxides of any of these metals can be used.

Noble metals (e.g., gold, silver, copper, platinum, palladium) are preferred for plasmon resonant particles, which are discussed in further detail below. For example, gold, silver, or an alloy comprising gold, silver, and optionally one or more other metals can be used. Core/shell particles (e.g., having a silver core with an outer shell of gold, or vice versa) can be used. Particles containing a metal core and a nonmetallic inorganic or organic outer shell, or vice versa, can be used. In certain embodiments, the nonmetallic core or shell comprises or consists of a dielectric material such as silica. Composite particles in which a plurality of metal particles are embedded or trapped in a nonmetal (e.g., a polymer or a silica shell) may be used. Hollow metal particles (e.g., hollow nanoshells) having an interior space or cavity are used in some embodiments. In some embodiments, a nanoshell comprising two or more concentric hollow spheres is used. Such a nanoparticle optionally comprises a core, e.g., made of a dielectric material.

In certain embodiments of the invention, at least 1%, or typically at least 5% of the mass or volume of the particle or number of atoms in the particle is contributed by metal atoms. In certain embodiments of the invention, the amount of metal in the particle, or in a core or coating layer comprising a metal, ranges from approximately 5% to 100% by mass, volume, or number of atoms, or can assume any value or range between 5 and 100%.

Certain metal nanoparticles, referred to as plasmon resonant particles, exhibit the well known phenomenon of plasmon resonance. When a metal nanoparticle (usually made of a noble metal such as gold, silver, copper, platinum, etc.) is subjected to an external electric field, its conduction electrons are displaced from their equilibrium positions with respect to the nuclei, which in turn exert an attractive, restoring force. If the electric field is oscillating (as in the case of electromagnetic radiation such as light), the result is a collective oscillation of the conduction electrons in the nanoparticle, known as plasmon resonance (68-70). The plasmon resonance phenomenon results in extremely efficient wavelength-dependent scattering and absorption of light by the particles over particular bands of frequencies, often in the visible range. Scattering and absorption give rise to a number of distinctive optical properties that can be detected using various approaches including visually (i.e., by the naked eye or using appropriate microscopic techniques) and/or by obtaining a spectrum, e.g., a scattering spectrum, extinction (scattering+absorption) spectrum, or absorption spectrum from the particle(s).

The features of the spectrum of a plasmon resonant particle (e.g., peak wavelength) depend on a number of factors, including the particle's material composition, the shape and size of the particle, the refractive index or dielectric properties of the surrounding medium, and the presence of other particles in the vicinity. Selection of particular particle shapes, sizes, and compositions makes it possible to produce particles with a wide range of distinguishable optically detectable properties thus allowing for concurrent detection of multiple RNAs by using particles with different properties such as peak scattering wavelenth.

Single plasmon resonant nanoparticles of sufficient size can be individually detected using a variety of approaches. For example, particles larger than about 30 nm in diameter are readily detectable under an optical microscope operating in dark-field. A spectrum from these particles can be obtained, e.g., using a CCD detector or other optical detection device. Despite their small dimensions relative to the wavelength of light, metal nanoparticles can be detected optically because they scatter light very efficiently at their plasmon resonance frequency. An 80 nm particle, for example, would be millions of times brighter than a fluorescein molecule under the same illumination conditions (69). Individual plasmon resonant particles can be optically detected using a variety of approaches including near-field scanning optical microscopy, differential interference microscopy with video enhancement, total internal reflection microscopy, photo-thermal interference contrast, etc. For measurements on a population of cells, a standard spectrometer, e.g., equipped for detection of UV, visible, and/or infrared light, can be used. In certain embodiments of the invention, nanoparticles are optically detected with the use of surface-enhanced Raman scattering (SERS) (71). Optical properties of metal nanoparticles and methods for synthesis of metal nanoparticles have been reviewed (72, 73).

Certain lanthanide ion-doped nanoparticles exhibit strong fluorescence and are of use in certain embodiments of the invention. A variety of different dopant molecules can be used. For example, fluorescent europium-doped yttrium vanadate (YVO₄) nanoparticles have been produced (74). These nanoparticles may be synthesized in water and are readily functionalized with biomolecules.

Magnetic nanoparticles are of use in the invention. “Magnetic particles” refers to magnetically responsive particles that contain one or more metals or oxides or hydroxides thereof. Such particles typically react to magnetic force resulting from a magnetic field. The field can attract or repel the particle towards or away from the source of the magnetic field, respectively, optionally causing acceleration or movement in a desired direction in space. A magnetically detectable nanoparticle is a magnetic particle that can be detected within a living cell as a consequence of its magnetic properties. Magnetic particles may comprise one or more ferrimagnetic, ferromagnetic, paramagnetic, and/or superparamagnetic materials. Useful particles may be made entirely or in part of one or more materials selected from the group consisting of: iron, cobalt, nickel, niobium, magnetic iron oxides, hydroxides such as maghemite (γ-Fe₂O₃), magnetite (Fe₃O₄), feroxyhyte (FeO(OH)), double oxides or hydroxides of two- or three-valent iron with two- or three-valent other metal ions such as those from the first row of transition metals such as Co(II), Mn(II), Cu(II), Ni(II), Cr(III), Gd(III), Dy(III), Sm(III), mixtures of the afore-mentioned oxides or hydroxides, and mixtures of any of the foregoing. See, e.g., U.S. Pat. No. 5,916,539 for suitable synthesis methods for certain of these particles. Additional materials that may be used in magnetic particles include yttrium, europium, and vanadium.

A magnetic particle may contain a magnetic material and one or more nonmagnetic materials, which may be a metal or a nonmetal. In certain embodiments of the invention, the particle is a composite particle comprising an inner core or layer containing a first material and an outer layer or shell containing a second material, wherein at least one of the materials is magnetic. Optionally both of the materials are metals. In one embodiment, the nanoparticle is an iron oxide nanoparticle, e.g., the particle has a core of iron oxide. Optionally the iron oxide is monocrystalline. In one embodiment, the nanoparticle is a superparamagnetic iron oxide nanoparticle, e.g., the particle has a core of superparamagnetic iron oxide.

Detection of magnetic nanoparticles may be performed using any method known in the art. For example, a magnetometer or a detector based on the phenomenon of magnetic resonance (NMR) can be employed. Superconducting quantum interference devices (SQUID), which use the properties of electron-pair wave coherence and Josephson junctions to detect very small magnetic fields can be used. Magnetic force microscopy or handheld magnetic readers can be used. U.S Pub. No. 2003/009029 describes various suitable methods. Magnetic resonance microscopy offers one approach (75).

In certain embodiments of the invention, the nanoparticle comprises a bulk material that is not intrinsically fluorescent, luminescent, plasmon resonant, or magnetic. The nanoparticle comprises one or more fluorescent, luminescent, or magnetic moieties. For example, the nanoparticle may comprise QDs, fluorescent or luminescent organic molecules, or smaller particles of a magnetic material. In some embodiments, an optically detectable moiety such as a fluorescent or luminescent dye, etc., is entrapped, embedded, or encapsulated by a nanoparticle core and/or coating layer.

In certain embodiments of the invention, the nanoparticle comprises silica (SiO₂). For example, the nanoparticle may consist at least in part of silica, e.g., it may consist essentially of silica or may have an optional coating layer composed of a different material. In some embodiments, the particle has a silica core and an outside layer composed of one or more other materials. In some embodiments, the particle has an outer layer of silica and a core composed of one or more other materials. The amount of silica in the particle, or in a core or coating layer comprising silica, can range from approximately 5% to 100% by mass, volume, or number of atoms, or can assume any value or range between 5% and 100%.

Silica-containing nanoparticles may be made by a variety of methods. Certain of these methods utilize the Stöber synthesis which involves hydrolysis of tetraethoxyorthosilicate (TEOS) catalyzed by ammonia in water/ethanol mixtures, or variations thereof. Microemulsion procedures can be used. For example, a water-in-oil emulsion in which water droplets are dispersed as nanosized liquid entities in a continuous domain of oil and surfactants and serve as nanoreactors for nanoparticle synthesis offer a convenient approach. Silica nanoparticles can be functionalized with biomolecules such as polypeptides and/or “doped” or “loaded” with certain inorganic or organic fluorescent dyes (see, e.g., U.S. Pub. No. 2004/0067503 and refs. 61-65).

In certain embodiments of the invention, the particle is made at least in part of a porous material, by which is meant that the material contains many holes or channels, which are typically small compared with the size of the particle. For example the particle may be a porous silica nanoparticle, e.g., a mesoporous silica nanoparticle or may have a coating of mesoporous silica (63). The particles may have pores ranging in diameter from about 1 nm to about 50 nm in diameter, e.g., between about 1 and 20 nm in diameter. Between about 20% and 95% of the volume of the particle may consist of empty space within the pores or channels.

In some embodiments of the invention, a nanoparticle composed in part or essentially consisting of an organic polymer is used. A wide variety of organic polymers and methods for forming nanoparticles therefrom are known in the art. For example, particles composed at least in part of polymethylmethacrylate, polyacrylamide, poly(vinyl chloride), carboxylated poly(vinyl chloride), or poly(vinyl chloride-co-vinyl acetate-co-vinyl alcohol) may be used. Optionally the nanoparticle comprises one or more plasticizers or additives. Co-polymers, block co-polymers, and/or grafted co-polymers can be used.

Fluorescent and luminescent moieties include a variety of different organic or inorganic small molecules commonly referred to as “dyes,” “labels,” or “indicators.” Examples include fluorescein, rhodamine, acridine dyes, Alexa dyes, cyanine dyes, etc. Fluorescent and luminescent moieties may include a variety of naturally occurring proteins and derivatives thereof, e.g., genetically engineered variants. For example, fluorescent proteins include green fluorescent protein (GFP), enhanced GFP, red, blue, yellow, cyan, and sapphire fluorescent proteins, reef coral fluorescent protein, etc. Luminescent proteins include luciferase, aequorin and derivatives thereof. Numerous fluorescent and luminescent dyes and proteins are known in the art (see, e.g., Valeur, B., “Molecular Fluorescence: Principles and Applications,” John Wiley and Sons, 2002; Handbook of Fluorescent Probes and Research Products, Molecular Probes, 9^{th} edition, 2002; and The Handbook—A Guide to Fluorescent Probes and Labeling Technologies, Invitrogen, 10^{th} edition, available at the Invitrogen web site).

In certain embodiments of the invention, the nanoparticle and the RNA are physically associated. Physical association can be achieved in a variety of different ways. The physical association may be covalent or non-covalent. The nanoparticle and the RNA may be directly linked to one another, e.g., by one or more covalent bonds, or may be linked by means of one or more linking agents. In one embodiment, the linking agent forms one or more covalent or non-covalent bonds with the nanoparticle and one or more covalent or non-covalent bonds with the RNA, thereby attaching them to one another. In some embodiments, a first linking agent forms a covalent or non-covalent bond with the nanoparticle and a second linking agent forms a covalent or non-covalent bond with the RNA. The two linking agents form one or more covalent or non-covalent bond(s) with each other. In some embodiments of the invention, the linkage to the nanoparticle will be to the material that forms a coating layer.

In some embodiments of the invention, the nanoparticle, the RNA, or both are linked to one or more additional moieties. The additional moiety can be a biomolecule such as a polypeptide, nucleic acid, polysaccharide, etc. Exemplary moieties include targeting agents (e.g., polypeptides that bind to a cell surface marker such as a cell surface receptor, translocation peptides, fusogenic or endosome disrupting peptides, etc.). The terms “polypeptide” and “peptide” are used interchangeably herein, with “peptide” typically referring to a polypeptide having a length of less than about 50 amino acids. Polypeptides may contain L-amino acids, D-amino acids, or both and may contain any of a variety of amino acid modifications or analogs known in the art. Useful modifications include, e.g., terminal acetylation, amidation, etc.

A variety of methods can be used to attach a biomolecule such as an RNA or polypeptide to a nanoparticle. General strategies include passive adsorption (e.g., via electrostatic interactions), multivalent chelation, high affinity non-covalent binding between members of a specific binding pair, covalent bond formation, etc. (67).

A bifunctional cross-linking reagent can be employed. Such reagents contain two reactive groups, thereby providing a means of covalently linking two target groups. The reactive groups in a chemical cross-linking reagent typically belong to various classes of functional groups such as succinimidyl esters, maleimides, and pyridyldisulfides. Exemplary cross-linking agents include, e.g., carbodiimides, N-hydroxysuccinimidyl-4-azidosalicylic acid (NHS-ASA), dimethyl pimelimidate dihydrochloride (DMP), dimethylsuberimidate (DMS), 3,3′-dithiobispropionimidate (DTBP), etc. For example, carbodiimide-mediated amide formation and active ester maleimide-mediated amine and sulfhydryl coupling are widely used approaches.

Common schemes for forming a conjugate involve the coupling of an amine group on one molecule to a thiol group on a second molecule, sometimes by a two- or three-step reaction sequence. A thiol-containing molecule may be reacted with an amine-containing molecule using a heterobifunctional cross-linking reagent, e.g., a reagent containing both a succinimidyl ester and either a maleimide, a pyridyldisulfide, or an iodoacetamide. Amine-carboxylic acid and thiol-carboxylic acid cross-linking, maleimide-sulfhydryl coupling chemistries (e.g., the maleimidobenzoyl-N-hydroxysuccinimide ester (MBS) method), etc., may be used. Polypeptides can conveniently be attached to nanoparticles via amine or thiol groups in lysine or cysteine side chains respectively, or by an N-terminal amino group. Nucleic acids such as RNAs can be synthesized with a terminal amino group. As described in Example 6, the inventors have employed a variety of coupling reagents (e.g., succinimidyl 3-(2-pyridyldithio)propionate (SPDP) and sulfosuccinimidyl-4-(N-maleimidomethyl)cyclohexane-1-carboxylate (sulfo-SMCC) to link QDs and siRNA or to link QDs and peptides. QDs can be prepared with functional groups, e.g., amine or carboxyl groups, available at the surface to facilitate conjugation to a biomolecule. Alternately, moieties such as biotin or streptavidin can be attached to the nanoparticle surface to facilitate binding to moieties functionalized with streptavidin or biotin, respectively.

Non-covalent specific binding interactions can be employed. For example, either the nanoparticle or the biomolecule can be functionalized with biotin with the other being functionalized with streptavidin. These two moieties specifically bind to each other non-covalently and with a high affinity, thereby linking the nanoparticle and the biomolecule. Other specific binding pairs could be similarly used. Alternately, histidine-tagged biomolecules can be conjugated to nanoparticles linked with nickel-nitrolotriaceteic acid (Ni-NTA).

Any biomolecule to be attached to a nanoparticle or RNA may include a spacer. The spacer can be, for example, a short peptide chain, e.g., between 1 and 10 amino acids in length, e.g., 1, 2, 3, 4, or 5 amino acids in length, a nucleic acid, an alkyl chain, etc.

In certain embodiments of the invention, a biomolecule is attached to a nanoparticle, or RNA via a cleavable linkage so that the biomolecule can be removed from the nanoparticle or RNA following intracellular delivery. In certain embodiments of the invention, a nanoparticle and an RNA (e.g., a short RNAi agent or tRNA) to be tracked or monitored in accordance with the invention may be conjugated to one another via a cleavable linkage so that the RNA can be released from the nanoparticle following cellular uptake. Removal or release can occur, for example, as a result of light-directed cleavage, chemical cleavage, protease-mediated cleavage, or enzyme-mediated cleavage. Cleavable linkages include disulfide bonds, acid-labile thioesters, etc. (90). Any linker that contains or forms such a bond could be employed. In one embodiment, the linker contains a polypeptide sequence that includes a cleavage site for an intracellular protease.

For additional general information on conjugation methods and cross-linkers, see the journal Bioconjugate Chemistry, published by the American Chemical Society, Columbus Ohio, PO Box 3337, Columbus, Ohio, 43210; “Cross-Linking,” Pierce Chemical Technical Library, available at the Pierce web site and originally published in the 1994-95 Pierce Catalog, and references cited therein; Wong S S, Chemistry of Protein Conjugation and Cross-linking, CRC Press Publishers, Boca Raton, 1991; and Hermanson, G. T., Bioconjugate Techniques, Academic Press, Inc., San Diego, 1996.

It is to be understood that the compositions of the invention can be made in any suitable manner, and the invention is in no way limited to compositions that can be produced using the methods described herein. Selection of an appropriate method may require attention to the properties of the particular moieties being linked.

If desired, various methods may be used to separate nanoparticles with an attached RNA, polypeptide, or other moiety from nanoparticles to which the moiety has not become attached, or to separate nanoparticles having different numbers of moieties attached thereto. For example, size exclusion chromatography or agarose gel electrophoresis can be used to separate populations of nanoparticles having different numbers of moieties attached thereto and/or to separate nanoparticles from other entities. Some methods include size-exclusion or anion-exchange chromatography.

As described further below, in some embodiments of the invention, one or more nanoparticles and one or more RNA molecules forms a non-covalent complex with a transfection reagent.

### IV. Delivery of Nanoparticles and RNA to Cells

Any of a variety of methods may be employed to deliver nanoparticle(s) and RNA to cells and/or to enhance delivery.

A. Transfection Reagents

Certain embodiments of the invention employ one or more transfection reagents to enhance intracellular delivery of a nanoparticle, RNA molecule, or both. The present invention demonstrates the formation of complexes comprising a transfection reagent, a nanoparticle, and an siRNA. Notably, the invention further demonstrates that such complexes can be efficiently delivered to the interior of mammalian cells and that the siRNA can effectively mediate gene silencing following internalization.

A variety of different transfection reagents are of use in the invention. A number of transfection reagents have been developed to enhance delivery of large DNA molecules (typically several hundred to thousands of base pairs in length), which differ significantly in terms of structure from small RNA species such as short RNAi agents and tRNAs. Nevertheless, certain of these transfection reagents mediate intracellular delivery of short RNAi agents and/or tRNAs.

A transfection reagent of use in the present invention may contain one or more naturally occurring, synthetic, and/or derivatized lipids. Cationic and/or neutral lipids or mixtures thereof may be used. Many cationic lipids are amphiphilic molecules containing a positively charged polar headgroup linked (e.g., via an anchor) to a hydrophobic domain often comprising two alkyl chains. Structural variations include the length and degree of unsaturation of the alkyl chains (86, 87). Cationic lipids include, for example, dimyristyl oxypropyl-3-dimethylhydroxy ethylammonium bromide (DMRIE), dilauryl oxypropyl-3-dimethylhydroxy ethylammonium bromide (DLRIE), N-[1-(2,3-dioleoyloxyl)propal]-n,n,n-trimethylammonium sulfate (DOTAP), dioleoylphosphatidylethanolamine (DOPE), dipalmitoylethylphosphatidylcholine (DPEPC), dioleoylphosphatidylcholine (DOPC), lipopolylysine, didoceyl methylammonium bromide (DDAB), 2,3-dioleoyloxy-N-[2-(sperminecarboxamidoethyl]-N,N-di-methyl-1-propanaminium trifluoroacetate (DOSPA), cetyltrimethylammonium bromide (CTAB), beta.-[N,(N′,N′-dimethylaminoethane)-carbamoyl]cholesterol (DC-Cholesterol, also known as DC-Chol), (-alanyl cholesterol, N-[1-(2,3-dioleoyloxy)propyl]-N,N,N-trimethylammonium chloride (DOTMA), N¹-cholesteryloxycarbonyl-3,7-diazanonane-1,9-diamine (CDAN), dipalmitoylphosphatidylethanolamine-5-carboxyspernylamide (DPPES), dicaproylphosphatidylethanolamine (DCPE), 4-dimethylaminopyridine (DMAP), dimyristoylphosphatidylethanolamine (DMPE), dioleoylethylphosphocholine (DOEPC), dioctadecylamidoglycyl spermidine (DOGS), and N-[1-(2,3-dioleoyloxy)propyl]-N-[1-(2-hydroxyethyl)]-N,N-dimethylammonium iodide (DOHME). Some representative cationic lipids include, but are not limited to, phosphatidylethanolamine, phospatidylcholine, glycero-3-ethylphosphatidyl-choline and fatty acyl esters thereof, di- and trimethyl ammonium propane, di- and tri-ethylammonium propane and fatty acyl esters thereof, e.g., N-[1-(2,3-dioleoyloxy)propyl]-N,N—,N-trimethylammonium chloride (DOTMA).

A variety of proprietary transfection reagents, most of which comprise one or more lipids, available commercially from suppliers such as Invitrogen (Carlsbad, Calif.), Quiagen (Valencia, Calif.), Promega (Madison, Wis.), etc., may be used. Examples include Lipofectin®, Lipofectamine®, Lipofectamine 2000®, Optifect®, Cytofectin®, Transfectace®, Transfectam®, Cytofectin®, Oligofectamine®, Effectene®, etc. A variety of transfection reagents have been developed or optimized for delivery of siRNA to mammalian cells. Examples include X-tremeGENE siRNA Transfection Reagent (Roche Applied Science), siIMPORTER™ siRNA Transfection Reagent (Upstate), BLOCK-iT™ Technology (Invitrogen), RNAiFect Reagent (QIAGEN), GeneEraser™ siRNA Transfection Reagent (Stratagene), RiboJuice™ siRNA Transfection Reagent (Novagen), EXPRESS-si Delivery Kit (Genospectra, Inc.), HiPerFect Transfection Reagent (QIAGEN), siPORT™, siPORT™ lipid, siPORT™ amine (all from Ambion), DharmaFECT™ (Dharmacon), etc.

Cationic polymers may be used as transfection reagents in the present invention. Exemplary cationic polymers include polyethylenimine (PEI), polylysine (PLL), polyarginine (PLA), polyvinylpyrrolidone (PVP), chitosan, protamine, polyphosphates, polyphosphoesters (see U.S. Pub. No. 2002/0045263), poly(N-isopropylacrylamide), etc. Certain of these polymers comprise primary amine groups, imine groups, guanidine groups, and/or imidazole groups. Some examples include poly(β-amino ester) (PAE) polymers (such as those described in U.S. Ser. Nos. 09/969,431 and 10/446,444; and U.S. Pub. No. 2002/0131951). The cationic polymer may be linear or branched. Blends, copolymers, and modified cationic polymers can be used. In certain embodiments of the invention, a cationic polymer having a molecular weight of at least about 25 kD is used. In one embodiment, deacylated PEI is used. For example, residual N-acyl moieties can be removed from commercially available PEI, or PEI can be synthesized, e.g., by acid-catalyzed hydrolysis of poly(2-ethyl-2-oxazoline), to yield the pure polycations (88).

Dendrimers are of use as transfection reagents in the present invention. Dendrimers are polymers that are synthesized as approximately spherical structures typically ranging from 1 to about 20 nanometers in diameter having a center from which chains extend in a tree-like, branching morphology. Molecular weight and the number of terminal groups increase exponentially as a function of generation (the number of layers) of the polymer. Different types of dendrimers can be synthesized based on different core structures. Dendrimers suitable for use with the present invention include, but are not limited to, polyamidoamine (PAMAM), polypropylamine (POPAM), polyethylenimine, iptycene, aliphatic poly(ether), and/or aromatic polyether dendrimers (see U.S. Pat. No. 6,471,968 and refs. 28 and 85).

Polysaccharides such as natural and synthetic cyclodextrins and derivatives and modified forms thereof are of use in certain embodiments of the invention (see, e.g., U.S. Pub. No. 2003/0157030 and ref. 82).

In certain embodiments of the invention, the transfection reagent forms a complex with one or more nanoparticles or RNAs. Typically the complex will contain a plurality of RNA molecules of one or more sequences, and a plurality of nanoparticles. Components of the complex are physical associated. The physical association is mediated, for example, by non-covalent interactions such as electrostatic interactions, hydrophobic or hydrophilic interactions, hydrogen bonds, etc., rather than covalent interactions or high affinity specific binding interactions. A complex can be formed when a moiety is encapsulated or entrapped by one or more other moieties. The present invention demonstrates that quantum dots, siRNA, and a transfection reagent can form a complex that is efficiently taken up by mammalian cells and that this uptake can be tracked and monitored by detecting the nanoparticles. Importantly, the invention demonstrates that the siRNA retains its gene silencing activity and that the signal detected from the internalized nanoparticles correlates with gene silencing activity.

Complex formation may take place by a variety of different mechanisms. For example, incubation of a lipid in the presence of RNA molecules and/or nanoparticles in an aqueous medium may result in formation of a liposome in which the RNA molecules and/or nanoparticles are encapsulated in an aqueous compartment. Alternatively or additionally, RNA molecules and/or nanoparticles may be entrapped in, or non-covalently associated with, the surface of the liposome. While not wishing to be bound by any theory, it is hypothesized that certain transfection reagents form a complex with the nanoparticles and RNA via electrostatic interactions. Liposomes formed from a lipid or combination thereof may be coated with a plurality of nanoparticles electrostatically attracted to the liposome surface.

Complexes can be formed by contacting a transfection reagent and nanoparticles for a period of time sufficient to allow complex formation to occur. The composition is then combined with RNA and the resulting composition is again maintained for a suitable period of time to allow complex formation to occur. Alternately, the transfection reagent and the RNA can first be allowed to form a complex, following which nanoparticles are combined with the composition. In one embodiment, the transfection reagent, nanoparticles, and RNA are mixed together and maintained for a suitable time period. Components can be combined by adding one to the other, by adding each of multiple components to a single vessel, etc. Suitable time periods for any of the afore-mentioned steps can be, e.g., several seconds, minutes, or hours (e.g., between 5-60 minutes or 10-30 minutes). Contacting typically takes place in an aqueous medium. A lipid transfection reagent may contain liposomes. In some embodiments, the liposomes are preformed liposomes. In some embodiments, other structures may form during the contacting. If desired, the physical characteristics of a complex comprising RNA molecules, nanoparticles, and a transfection reagent can be evaluated using a variety of methods known in the art. For example, the size, charge, and/or polydispersity of the complex can be determined using a Malvern Instruments Zetasizer (Malvern, UK), dynamic light scattering, etc.

Standard transfection protocols can be used to deliver the RNA and nanoparticles to cells. Typically the cells are contacted with the transfection reagent, nanoparticles, and RNA (e.g., as a complex) for time periods ranging from minutes to hours. Protocols can be varied to optimize uptake.

The invention encompasses the use of magnetic forces to enhance uptake of nanoparticles, RNA, or both, by cells. In one embodiment, a complex comprises a magnetic nanoparticle and an siRNA.

B. Electroporation

In certain embodiments of the invention, an electric field is applied to enhance intracellular delivery of a nanoparticle sensor component. Application of an electric field to cells to enhance their uptake of DNA, a technique referred to as electroporation, has long been known in the art (83, 84). While not wishing to be bound by any theory, the mechanism may involve temporary disruption of the cell membrane, allowing foreign bodies to enter, followed by resealing of the membrane. In the present invention electroporation is used to enhance the uptake of RNA and nanoparticles by cells. Standard electroporation protocols known in the art can be used. Parameters such as electric field strength, voltage, capacitance, duration and number of electric pulse(s), cell number of concentration, and the composition of the solution in which the cells are maintained during or after electroporation can be optimized for the delivery of RNA and of nanoparticles of any particular size, shape, and composition and/or to achieve desired levels of cell viability. The methods of the invention are not limited to parameters that have been successfully used to enhance cell transfection in the art. Exemplary parameter ranges include, e.g., charging voltages of 100-500 volts and pulse lengths of 0.5-20 ms.

C. Microinjection

In certain embodiments of the invention, cells are microinjected with a composition comprising an RNA and an optically or magnetically detectable nanoparticle. Optionally the RNA and the nanoparticle are physically associated. An automated microinjection apparatus can be used (see, e.g., U.S. Pat. No. 5,976,826).

D. Translocation Peptides

In certain embodiments of the invention, the transfection reagent comprises a translocation peptide. The translocation peptide can be any of a variety of protein domains that are capable of inducing or enhancing translocation of an associated moiety into a eukaryotic cell, e.g., a mammalian cell. For example, presence of these domains within a larger protein enhances transport of the larger protein into cells. These domains are sometimes referred to as protein transduction domains (PTDs) or cell penetrating peptides (CPPs). Translocation peptides include peptides derived from various viruses, DNA binding segments of leucine zipper proteins, synthetic arginine-rich peptides, etc. (see, e.g., Langel, U. (ed.), Cell-Penetrating Peptides Processes and Applications, CRC Press, Boca Raton, Fla., 2002).

Exemplary translocation peptides that may be used in accordance with the present invention include, but are not limited to, the TAT₄₉₋₅₇ peptide, referred to herein as “TAT peptide” (sequence: RKKRRQRRR (SEQ ID NO: 1)) from the HIV-1 protein (95, 96); longer peptides that comprise the TAT peptide; and the peptide RQIKIWFZQRRMKWKK (SEQ ID NO: 2) from the Antennapedia protein.

In some embodiments, translocation-enhancing moieties of use include peptide-like molecules known as peptoid molecular transporters (U.S. Pat. Nos. 6,306,933 and 6,759,387). Certain of these molecules contain contiguous, highly basic subunits, particularly subunits containing guanidyl or amidinyl moieties.

E. Endosome Escape Agents

In some embodiments of the invention, an endosome disrupting or fusogenic agent is administered to cells to enhance release of nanoparticles, RNA, or both from the endosome. Examples include fusogenic peptides, chloroquine, various viral components such as the N-terminal portion of the influenza virus HA protein (e.g., the HA2 peptide), adenoviral proteins or portions thereof, etc. (see, e.g., U.S. Pat. No. 6,274,322). For example, in certain embodiments of the invention, the endosome disrupting agent is a peptide comprising the N-terminal 20 amino acids of the influenza HA protein. In some embodiments, the INF-7 peptide, which resembles the NH₂-terminal domain of the influenza virus hemagglutinin HA-2 subunit, is used. In certain embodiments of the invention, an endosome escape agent or fusogenic peptide is conjugated to the nanoparticle, the RNA, or both.

The membrane-lytic peptide mellitin may be used. In certain embodiments of the invention, an endosome disrupting agent is conjugated to an RNA, a nanoparticle, or both. In certain embodiments of the invention, a polypeptide having a first domain that serves as an endosome disrupting or fusogenic agent and a second domain that serves as a translocation peptide is employed. An agent that enhances release of endosomal contents or escape of an attached moiety from an internal cellular compartment such as an endosome may be referred to as an “endosomal escape agent.”

F. Targeted Nanoparticles

In certain embodiments of the invention, the nanoparticle comprises a targeting agent. A targeting agent is any agent that binds to a component present on or at the surface of a cell. Such a component is referred to as a “marker.” The marker can be a polypeptide or portion thereof. The marker can be a carbohydrate moiety. The marker can be cell type specific, disease state specific, etc. For example, the marker may be expressed in significant amounts mainly on one or a few cell types or in one or a few diseases. A cell type specific marker for a particular cell type is expressed at levels at least 3 fold greater in that cell type than in a reference population of cells which may consist, for example, of a mixture containing cells from a plurality (e.g., 5-10 or more) of different tissues or organs in approximately equal amounts. In some embodiments, the cell type specific marker is present at levels at least 4-5 fold, between 5-10 fold, or more than 10-fold greater than its average expression in a reference population. Detection or measurement of a cell type specific marker may make it possible to distinguish the cell type or types of interest from cells of many, most, or all other types.

Numerous markers are known in the art. Typical markers include cell surface proteins, e.g., receptors. Exemplary receptors include, but are not limited to, the transferrin receptor; LDL receptor; growth factor receptors such as epidermal growth factor receptor family members (e.g., EGFR, HER-2, HER-3, HER-4, HER-2/neu) or vascular endothelial growth factor receptors; cytokine receptors; cell adhesion molecules; integrins; selectins; CD molecules; etc. The marker can be a molecule that is present exclusively or in higher amounts on a malignant cell, e.g., a tumor antigen. For example, prostate-specific membrane antigen (PSMA) is expressed at the surface of prostate cancer cells. In certain embodiments of the invention the marker is an endothelial cell marker.

The targeting agent may be a polypeptide, peptide, nucleic acid, carbohydrate, glycoprotein, lipid, small molecule, etc. For example, the targeting agent may be a naturally occurring or synthetic ligand for a cell surface receptor, e.g., a growth factor, hormone, LDL, transferrin, etc. The targeting agent can be an antibody, which term is intended to include antibody fragments, single chain antibodies, etc. Synthetic binding proteins such as affibodies, etc., can be used. Peptide targeting agents can be identified, e.g., using procedures such as phage display. This widely used technique has been used to identify cell specific ligands for a variety of different cell types. In certain embodiments of the invention, the ligand is an aptamer that binds to a cell type specific marker. In general, an aptamer is an oligonucleotide (e.g., DNA or RNA or an analog thereof) that binds to a particular target, such as a polypeptide. Aptamers are typically derived from an in vitro evolution process such as SELEX, and methods for obtaining aptamers specific for a protein of interest are known in the art.

In certain embodiments of the invention the marker is a tumor marker. The marker may be a polypeptide that is expressed at higher levels on dividing than on non-dividing cells. Nucleolin is an example. The peptide known as F3 is a suitable targeting agent for directing a nanoparticle to nucleolin (92, 93). As described in Example 6, the inventors demonstrated that conjugating nanoparticles (QDs) with peptide F3 improved nanoparticle uptake by tumor cells.

It will be appreciated that various changes in the amino acid sequence of a peptide, such as an endosome disrupting peptide, translocation peptide, cell targeting peptide, etc., can be made without substantially affecting the ability of the peptide to enhance endosome escape. For example, 1, 2, 3, or more such changes such as deletions, insertions, substitutions, etc. may be made. Typically the resulting peptide will have at least 80% sequence identity, e.g., 90% sequence identity, with the original peptide. Such variations are within the scope of the invention.

FIG. 10 presents a schematic diagram illustrating multifunctional nanoparticles for siRNA delivery in one embodiment. The particles, which are optionally optically or magnetically detectable, contain a core and a coating layer. The surface of the particles is functionalized with a targeting peptide, an endosomal escape peptide, and an siRNA. The targeting agent binds to a cell surface marker that is selectively present on malignant cells. The particle is internalized and enters the endosome. The siRNA is released from the particle, optionally as a result of cleavage of a labile bond such as a disulfide, and the siRNA is released from the endosome into the cytoplasm, where it silences a gene in a therapeutically useful manner. The optically or magnetically detectable nanoparticle can be detected to provide an indication of cellular uptake of the siRNA and/or its gene silencing activity. The method thus facilitates evaluating the efficacy of different siRNAs, different delivery vehicles, etc. The method is of use to guide dosing for therapy of a disease that is treated by the siRNA.

### V. Cells

The invention may be used to track and monitor uptake of RNA by any eukaryotic cell of interest. In certain embodiments of the invention, the cell is a mammalian cell. The cells may be of human or non-human origin. For example, they may be of mouse, rat, or non-human primate origin. The cell can be of any cell type. Exemplary cell types include, but are not limited to, endothelial cells, epithelial cells, neurons, hepatocytes, myocytes, chondrocytes, osteoblasts, osteoclasts, lymphocytes, macrophages, neutrophils, fibroblasts, keratinocytes, etc. The cells can be primary cells, immortalized cells, transformed cells, terminally differentiated cells, stem cells (e.g., adult or embryonic stem cells, hematopoietic stem cells), somatic cells, germ cells, etc. The cells can be wild type or mutant cells, e.g., they may have a mutation in one or more genes. The cells may be quiescent or actively proliferating. The cells may be in any stage of the cell cycle.

The cells can be normal cells or diseased cells. In certain embodiments of the invention, the cells are cancer cells, e.g., they originate from a tumor or have been transformed in cell culture (e.g., by transfection with an oncogene). In certain embodiments of the invention, the cells are infected with a virus or other infectious agent. The virus may be, e.g., a DNA virus, RNA virus, retrovirus, etc. For example, the cells can be infected with a human pathogen such as a hepatitis virus, a respiratory virus, human immunodeficiency virus, etc.

The cells may have been experimentally manipulated to overexpress one or more genes of interest, e.g., by transfecting them with an expression vector that contains a coding sequence operably linked to expression signal(s) such as a promoter.

The cells can be cells of a cell line. Exemplary cell lines include HeLa, CHO, COS, BHK, NIH-3T3, HUVEC, etc. For an extensive list of mammalian cell lines, those of ordinary skill in the art may refer to the American Type Culture Collection catalog (ATCC®, Manassas, Va.).

Cell Sorting and Processing

In addition to detection of nanoparticle(s) within cells, the invention provides methods in which cells are optionally analyzed, sorted, and/or manipulated in any of a variety of ways. For example, after a collection of cells has been contacted with a nanoparticle and an RNA, the collection of cells can be separated into two or more populations (sorted), e.g., based on an optical or magnetic signal acquired from individual cells, which reflects the number of nanoparticles contained in the cells.

A variety of different methods for analyzing and separating cells can be used. For example, flow cytometers capable of sorting cells on the basis of their fluorescence characteristics (fluorescence activated cell sorting (FACS)) can be used (see, e.g., Ormerod, M. G., Flow Cytometry: A Practical Approach, 3^{rd} ed., Oxford University Press, 2000). Flow cytometry may separate cells based on simultaneous in-line video microscopy, which can detect a variety of different cellular parameters (76). Magnetic cell sorting can be employed (78).

Cells can be selected for manipulation or processing based on their optical or magnetic properties following nanoparticle internalization. In certain embodiments, cells are physically manipulated. Suitable methods for physically manipulating single cells include, e.g. manipulation techniques such as optical tweezers, electrokinetic forces (electrophoresis, dielectrophoresis, traveling-wave dielectrophoresis), magnetic tweezers, acoustic traps and hydrodynamic flows. In one embodiment, an incoherent light source (a light-emitting diode or a halogen lamp) and a digital micromirror spatial light modulator are employed, offering a highly parallel system capable of manipulating thousands of cells (81).

In certain embodiments of the invention, processing procedures are performed. For example, cells identified as having taken up an undesirably small or large RNA can be eliminated from a population. In one embodiment, a scanning cytometer with laser ablation is employed to ablate particular cells. Suitable instruments are available, e.g., from Cyntellect, Inc. (San Diego, Calif.).

In certain embodiments of the invention, detection and, optionally, sorting, manipulation, ablation, etc., is accomplished in a microfluidic device. A variety of microfluidic devices that incorporate detection capabilities, and, optionally, fluid manipulation, sorting, and other capabilities are known in the art. Such devices are sometimes referred to as a “lab-on-a-chip.” An exemplary microfluidic cell sorter is described in U.S. Pat. No. 6,540,895. The inventive methods may be used to sort cells into different chambers of a microfluidic device. In one embodiment, microfluidic sorting of cells is accomplished using optical force switching (79). In one embodiment, gravity and electric force driving of cells are used to perform flow cytometry and fluorescence activated cell sorting in a microfluidic chip system (80).

Additional processing can include exposing cells to compounds. For example, cells that have been contacted with an siRNA that silences a particular gene may be exposed to a compound to determine whether the compound has an effect on the cell in the absence of the gene product. Such experiments may be useful, for example, to identify targets of drug activity.

Of course cells can simply be observed, analyzed, and/or compared using any method known in the art following cell selection and/or separation into different populations. Any cellular feature, characteristic, or behavior can be compared. For example, cell migration, cell proliferation, cell death, etc., can be assessed. Additional experiments such as measuring the level of any particular mRNA or protein of interest can be performed on one or more cells or populations of cells using standard methods. In one embodiment, a feature, characteristic, or behavior of cells that have taken up a large amount of an siRNA can be compared with that of cells that have taken up a lesser amount of RNA.

### VI. Kits and Related Methods

The invention provides a variety of kits. The kits may include one or more optically or magnetically detectable nanoparticles. For example, the kits may include 1, 2, 3, 4, or more nanoparticles having distinguishable optical and/or magnetic properties. In one embodiment, the kits include a collection of different QDs having different peak emission wavelengths. For example, the kits may include QDs having peak emission wavelengths selected from the group consisting of approximately 525, 535, 545, 565, 585, 605, 655, 705, and 800 nm. Typically the kits will include sufficient amounts of QDs to allow the user to perform multiple experiments. The nanoparticles may be functionalized, e.g., with a translocation peptide, an endosome escape peptide, a targeting agent, etc.

The kits may include additional components or reagents. For example, the kits may include one or more transfection reagents, e.g., any of the transfection reagents described herein. The kits may include one or more RNAs, e.g., a control RNA. The kits may include a translocation peptide, an endosome escape peptide, a targeting agent, etc. The kits may include a cross-linking agent, linker, or any other component that could be used to conjugate a nanoparticle or RNA to a biomolecule. The kits may include cells and/or cell culture medium.

In some embodiments, the kit is supplied with or includes one or more RNAs, e.g., siRNAs, specified by the purchaser.

The kit may include instructions for use. For example, the instructions may inform the user of the proper procedure by which to prepare a complex comprising a transfection reagent, nanoparticles, and RNA molecules and/or the proper procedure for contacting cells with the nanoparticles, RNA, transfection reagent, etc.

Kits may include one or more vessels or containers so that certain of the individual components or reagents may be separately housed. The kits may include a means for enclosing the individual containers in relatively close confinement for commercial sale, e.g., a plastic box, in which instructions, packaging materials such as styrofoam, etc., may be enclosed.

The invention provides a method of supplying an RNAi agent comprising steps of: (a) electronically receiving an order for an RNAi agent from a requester; and (b) providing the RNAi agent and an optically or magnetically detectable nanoparticle to the requester. Typically the order may include a request for the optically or magnetically detectable nanoparticle or an indication that the requester desires to be supplied with the particle.

The invention provides a method of supplying a nanoparticle comprising steps of: (a) electronically receiving an order for an optically or magnetically detectable nanoparticle from a requestor; and (b) providing the optically or magnetically detectable nanoparticle and an RNAi agent to the requestor. Typically the order includes a request for the RNAi agent or an indication that the requestor desires to be supplied with the RNAi agent.

The invention further provides a method of placing an order for an optically or magnetically detectable nanoparticle and an RNAi agent comprising steps of: (a) electronically creating or transmitting an order for an RNAi agent and an optically or magnetically detectable nanoparticle from a supplier.

It will be understood that in any of these methods, the nanoparticle and/or RNAi agent is requested or provided for use in a composition or method of the invention, e.g., to in order to track or monitor uptake of the RNAi agent by cells. A nanoparticle or RNAi agent will be considered to be requested for use in a composition or method of the invention if the requestor places the order with the intent of so using the nanoparticle or RNAi agent and/or does so use the nanoparticle or RNAi agent. A nanoparticle or RNAi agent will be considered to be supplied for use in a composition or method of the invention if (i) the supplier supplies the nanoparticle and the RNAi agent with instructions for their use in practicing a method of the invention or instructions for preparing a composition of the invention and/or (ii) the supplier advertises or promotes the use of an RNAi agent and an optically or magnetically detectable nanoparticle to practice a method of the invention or to prepare a composition of the invention or provides instructions for such use in any manner. The invention encompasses advertising or promoting or providing instructions for the practice of a method of the invention or the preparation of a composition of the invention, regardless of whether material(s) for practicing the invention may be provided.

The terms “electronically receiving,” “order,” “requester,” and “providing” are to be interpreted broadly and without limitation. For example, “electronically receiving” can refer to any method by which information can be received that involves electronic means for the creation, transmission, and/or receipt of the order. For example, “electronically receiving” can mean by phone, by fax, by computer (e.g., by e-mail, by submitting an “order form” over the Internet), etc. The transmission and receipt of the order can be wireless. “Electronically receiving” can mean receiving, by mail, a computer-readable medium having information stored thereon. An “order” is any means by which a request can be made. A “requester” is any individual or entity that seeks to obtain an item. “Providing” means any means of supplying an item such as an RNAi agent, nanoparticle, etc. For example, “providing” can refer to sending the item to a destination or arranging for the item to be sent. Any means of sending may be employed. It will be understood that the RNAi agent and the nanoparticle are typically supplied within a single container such as a box containing smaller receptacles to house the RNAi agent and the nanoparticle. However, the two items can be supplied separately. It will be understood that the RNAi agent and the nanoparticle are typically supplied in a temporal relationship with one another, e.g., they are either sent together in a single container or are sent separately within 24-48 hours of one another. The methods may further include providing any of the items that may be present in a kit, as described above.

Typically, the RNAi agent is a short RNAi agent such as an siRNA. In an exemplary embodiment, a user, e.g., a researcher, desiring to employ an inventive method for tracking or monitoring delivery of an siRNA to eukaroytic cells, submits an order for an siRNA targeted to a particular gene over the Internet (e.g., by filling out and submitting a Web-based form). The researcher may submit an order for an optically or magnetically detectable nanoparticle such as a QD. The supplier receives the order and ships the siRNA and the QD to the researcher.

### VII. Applications

The inventive methods for tracking and monitoring RNA and/or its activity have a wide variety of uses, such as those mentioned above and in the examples. This section provides additional details regarding particular applications of the compositions and methods. The inventive methods may be used to compare the silencing activity of different siRNAs or other short RNAi agents towards a target gene. The methods provide a means of normalizing for siRNA uptake, thereby controlling for this variable, to provide a more accurate reflection of the intrinsic silencing activity of a particular siRNA. Thus the invention provides a method of testing an RNAi agent comprising steps of: contacting a cell with a detectable nanoparticle and an RNAi agent designed to silence a gene; detecting the nanoparticle; and determining the silencing activity of the RNAi agent towards the gene. The method can comprise contacting first and second cells with first and second RNAi agents designed to silence the same gene and comparing the silencing activity of the first and second RNAi agents after normalizing for the amount of each RNAi agent taken up by the cell(s) with which it is contacted.

The inventive methods may be used to compare the ability of different delivery vehicles to facilitate siRNA delivery to cells in culture or in vivo. The delivery vehicle may, but need not be, a transfection reagent such as those described herein. Other delivery vehicles, carriers, etch, are within the scope of the invention. In one embodiment, the invention provides a method of testing a delivery vehicle comprising contacting a cell with a detectable nanoparticle, an RNAi agent designed to silence a gene, and a delivery vehicle; detecting the nanoparticle; and determining the silencing activity of the RNAi agent towards the gene. The method can comprise contacting a first cell with a first delivery vehicle, a detectable nanoparticle, and an RNAi agent designed to silence a gene; contacting a second cell with a second delivery vehicle, a detectable nanoparticle, and an RNAi agent designed to silence a gene; and comparing silencing of the gene in the first and second cells or comparing amounts of the detectable nanoparticle in the first and second cells.

The invention encompasses in vivo applications of the compositions and methods described herein. In certain embodiments of the invention, a composition comprising a detectable nanoparticle, e.g., a QD, and an RNAi agent (e.g., an siRNA) is administered to a subject. Any of the detectable nanoparticles described herein may be used. For example, in some embodiments, the nanoparticle and the RNAi agent are conjugated to one another. In one embodiment, an additional moiety such as a translocation peptide is conjugated to the nanoparticle. The in vivo applications encompass administering multiple nanoparticles having distinguishable properties, each associated with a different RNAi agent, to a subject, providing the ability to track and monitor silencing of multiple genes.

The subject may be, for example, an animal such as a mouse, rat, non-human primate, or other animal used as a model for human disease. The subject to whom the composition is administered may be a human being. A variety of routes of administration can be employed including, but not limited to parenteral (e.g., intravenous, intraarterial, intramuscular, subcutaneous injection), oral (e.g., dietary), inhalation (e.g., aerosol to lung), topical or transdermal, nasal, vaginal, buccal, rectal, etc.). In one embodiment, conventional volumes and injection times are employed for intravenous administration. In one embodiment, the technique known as hydrodynamic transfection is used to deliver a composition to a small animal such as a mouse. The composition may comprise a delivery vehicle. The delivery vehicle may be specifically adapted for delivery of an RNAi agent such as an siRNA. The composition may comprise any carrier, diluent, excipient, or other component known in the art for use in a composition to be delivered to a subject, e.g., for use in a pharmaceutical composition.

Following administration to the subject the nanoparticle is detected, thereby providing an indication of the distribution and/or uptake of the RNAi agent by various cells, tissues, organs, etc., and optionally providing an indication of the silencing activity of the RNAi agent in such cells, tissues, organs, etc. Detection can take place at any suitable time following administration. In one embodiment, a tissue sample (e.g., a tissue section) is obtained from the subject and examined microscopically by any of the techniques described herein. Alternately, individual cells can be isolated from the subject and examined, sorted, or further processed. In vivo imaging techniques such as fluorescence imaging can be employed to detect nanoparticles in a living subject (27). In vivo administration provides the potential for rapidly evaluating the ability of different delivery vehicles to enhance siRNA uptake in a living organism, thereby facilitating efforts to develop therapeutic agents comprising siRNAs. In addition to detecting nanoparticles, conventional immunostaining or other techniques can be employed, e.g., to confirm gene silencing activity of an RNAi agent, to gather information about the effect of gene silencing by the RNAi agent on the subject, etc.

In addition to monitoring the uptake and/or presence of RNA in cells, the invention can be used to track and/or quantitate an isolated composition comprising one or more RNA species. For example, a first RNA (e.g., a first siRNA) and a first nanoparticle population can be combined to form a first composition. A second RNA (e.g., a second siRNA) and a second nanoparticle population can be combined to form a second composition. When aliquots from the first and second compositions are mixed to form a third composition, the optical or magnetic signals from the first and second nanoparticle populations are indicative of the amounts of the first and second RNAs in the third composition. The ratio of the two signals is indicative of the ratios of the first and second RNAs in the third composition.

The signal may be conveniently obtained using any suitable approach. For example, an emission, absorption, or scattering spectrum can be obtained from a solution containing one or more RNAs and corresponding nanoparticle population(s).

The method can be extended to any number of RNAs, each having a corresponding nanoparticle population. It can be employed simply to track or monitor the concentration or amount of a single RNA, e.g., through multiple manipulations or reactions.

This approach provides a convenient means of quantifying RNA species and/or monitoring the RNA concentration in a composition through multiple manipulations or reactions without the need to modify the RNA. The method may be used, for example, in conditions where the RNA concentration is expected to be very low such that conventional means of measuring RNA concentration would be inaccurate, or in the presence of substances that would interfere with conventional methods for RNA measurement.

### EXEMPLIFICATION

### Example 1

### Co-Delivery of Quantum Dots and siRNA to Cells Allows Quantitation of siRNA Uptake and Correlation of Gene Silencing with Intracellular Fluorescence

### Materials and Methods

Short Interfering RNA and Quantum Dot Preparation

Pre-designed siRNA was used to selectively silence the Lamin A/C gene (Lmna siRNA #73605, NM_{—}019390, Ambion) and the T-cadherin gene (SMARTpool reagent CDH13, NM_{—}019707, Dharmacon). Fluorescently-labeled Lmna siRNA purchased from Dharmacon was designed with a fluorescein molecule on the 5′ end of the sense strand. The annealed sequences were reconstituted in nuclease-free water and used at a concentration of 100 nM (Lmna siRNA, 5′-Fluorescein-Lmna siRNA) or 50 nM (T-cad siRNA).

Green (560 nm emission maxima) and orange (600 nm emission maxima) CdSe-core, ZnS-shell nanocrystals were synthesized and water-solubilized with mercaptoacetic acid (MAA) as previously described (29-31). MAA-QDs were then surface-modified by reacting with polyethylene glycol (PEG)-thiol MW 5000 (Nektar) overnight at room temperature. Excess PEG-thiol was removed by spin filtration (100 kDa cutoff). QDs are also available commercially as an alternative to synthesis (Quantum Dot Corporation, Evident Technologies). Unless stated otherwise, 5 μg PEGylated QD was used per cell transfection.

Fibroblast Cell Culture and Transfection

3T3-J2 fibroblasts were provided by Howard Green (Harvard Medical School, Cambridge, Mass.) (32) and cultured at 37° C., 5% CO₂ in Dulbecco's Modified Eagle Medium (DMEM) with high glucose, 10% fetal bovine serum (FBS), and 1% penicillin-streptomycin. The transfection procedure was performed using Lipofectamine 2000 (Invitrogen) according to the manufacturer's instructions. Briefly, 3T3 fibroblasts were plated 24 h prior to transfection at a density of 3×10⁶ cells per 35-mm well, in antibiotic- and serum-free medium. Lipofectamine reagent (5 μl) and either siRNA or QDs were diluted in Dulbecco's Modified Eagles' Medium (DMEM) and complexed at room temperature. For QD/siRNA co-complexes, siRNA and liposomes were allowed to complex for 15 min prior to an additional 15 min incubation with QDs. Complexes were added to cell cultures in fresh antibiotic- and serum-free medium until 5 hours later, at which time the cultures were washed and replaced with regular growth medium. Approximately 24 hours post-transfection, cells were trypsinized and prepared for flow cytometry.

Fluorescence Activated Cell Sorting (FACS)

Flow cytometry and sorting was performed on a FACS Vantage SE flow cytometer (Becton Dickinson) using a 488 nm Ar laser and FL1 bandpass emission (530±20 nm) for the green QDs, FL3 bandpass emission (610±10 nm) for the orange QDs. Fluorescence histograms and dot plots were generated using Cell Quest software (for figures, histograms were re-created using WinMDI software, Scripps Institute, CA). Cell Quest was also used to gate populations of highest and lowest fluorescence intensity for sorting into chilled FBS. Sorted populations were immediately re-plated into separate wells containing regular growth medium and allowed to adhere. Cells were incubated at 37° C. until visualized by fluorescence microscopy or until assayed for protein level.

Western Blotting

Cell cultures were scraped and lysed in RIPA Lysis Buffer (Upstate Biotechnologies) supplemented with COMPLETE EDTA-free Protease inhibitor solution (Roche). Equal amounts (15-20 μg) total protein were loaded onto a 10% Tris-HCl resolving gel, separated by electrophoresis, and transferred to PVDF membrane. The blot was incubated in blocking solution (5% [w/v] nonfat dry milk, 200 mM Tris base [pH 7.4], 5 M NaCl, 5% Tween-20) for 1 hour at room temperature, primary antibody overnight at 4° C., and secondary antibody for 1 hour. Three washes in 200 mM Tris base pH 7.4, 5 M NaCl, 5% Tween-20 took place between steps and after completion of probing. Finally, the blot was visualized by chemiluminescence (Super Signal West Pico Kit, Pierce) and developed. Bands were analyzed for density using MetaMorph Image Analysis software (Universal Imaging) and normalized to loading control (β-actin) bands.

Primary antibodies used were polyclonal lamin A/C antibody (Cell Signaling) at 1:1000 dilution in blocking solution and polyclonal β-actin antibody (Cell Signaling) at 1:750 dilution. T-cadherin primary antibody was a gift from Barbara Ranscht (University of California, San Diego) (33). Secondary antibody was goat anti-rabbit IgG-HRP (Santa Cruz Biotechnology) at 1:7500 dilution. Blots were probed simultaneously for lamin A/C protein (70 kDa, 28 kDa) and β-actin protein (45 kDa); after detection, select blots were re-probed for T-cadherin (95 kDa).

Immunofluorescence Staining

Sorted and unsorted cells intended for lamin nuclear protein immunostaining were seeded onto Collagen-I coated glass coverslips. Coverslips with attached cells were washed twice in cold phosphate-buffered saline (PBS, Gibco) and fixed in 4% paraformaldehyde at room temperature. After three brief PBS washes, cells were permeabilized with 0.2% Triton-X for 10 min at room temperature and washed again. The cells were blocked with 10% goat serum for 30 min at 37° C., incubated in primary antibody (1:100 Lamin A antibody, Santa Cruz Biotechnology) for 90 min at 37° C., washed three times with 0.05% Triton-X, incubated in secondary antibody (1:250 AlexaFluor 594 chicken anti-rabbit IgG antibody, Molecular Probes) for 1 hour at room temperature, and washed a final three times. Antibody dilutions were performed in 1% bovine serum albumin (BSA) in PBS. Coverslips were mounted onto glass slides using Vectashield anti-fade medium (Vector Laboratories). Finally, nuclear staining was visualized and documented by phase contrast microscopy or epifluorescence (Nikon Ellipse TE200 inverted fluorescence microscope and CoolSnap-HQ Digital CCD Camera).

### Results

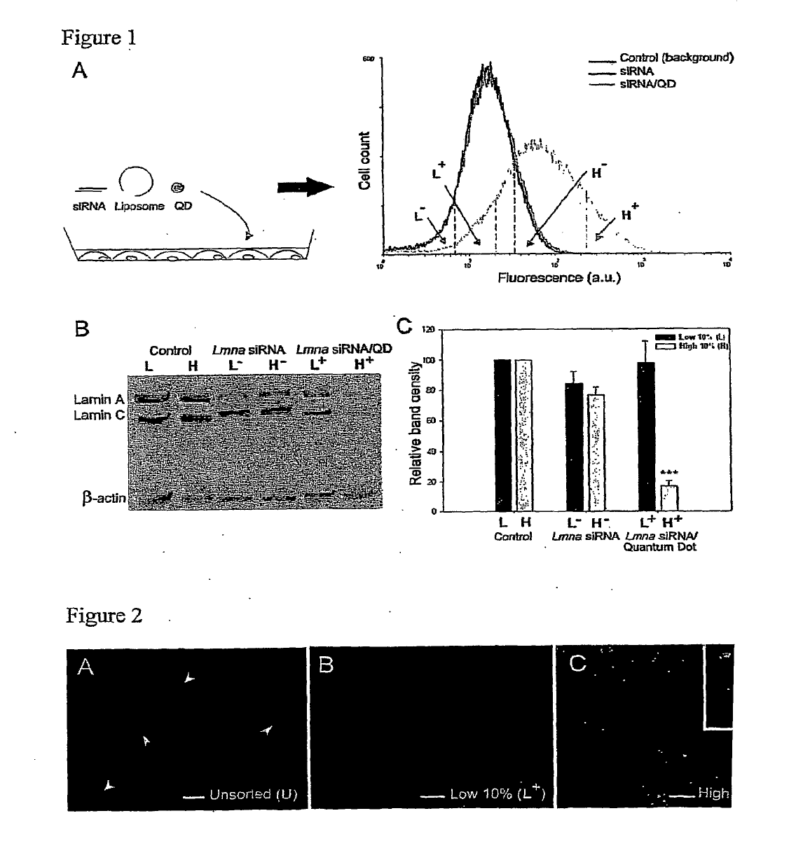

We used cationic liposomes to co-deliver green QDs and siRNA targeting the lamin A/C gene (Lmna) into murine fibroblasts, followed by flow cytometry to quantify intracellular QD uptake (FIG. 1A). The median fluorescence of QD/siRNA-transfected cells compared to mock-transfected cells (liposome reagent only) and cells transfected with siRNA alone varied by approximately 84% (coefficient of variation). FACS was used to gate and collect the brightest 10% (high, H) of each fluorescence distribution, along with the dimmest 10% (low, L).

After the sorted cells were re-plated and grown for 72 hours to ensure protein turnover, protein expression analysis by either Western blot or immunostaining was performed. In cells that had been co-transfected with siRNA and QDs, gene silencing correlated directly with intracellular fluorescence. Western blotting (FIG. 1B) and image analysis of lamin A/C protein bands (FIG. 1C) show approximately 90% knockdown in the highly fluorescent cells and negligible knockdown in the dimmest cells. The cells treated with siRNA alone exhibited mediocre gene down-regulation (20-30%) independent of sorting parameters. Consistent with the quantitative bulk protein assay, immunofluorescent detection of lamin nuclear protein in unsorted, siRNA-transfected cells produced heterogeneous staining throughout the cell population (FIG. 2A). However, in the co-transfected case, the presence of green QDs correlated with consistently weak lamin immunofluorescent staining in the high co-transfected subpopulation (FIG. 2B), compared to a lack of observable QDs and strong lamin staining in the low subpopulation (FIG. 2C). Heterogeneous silencing therefore influences the accuracy of the bulk protein expression readout, suggesting the importance of verifying successful siRNA transfection for each gene knockdown study. Using QDs as photostable probes in combination with FACS, a subpopulation of uniformly-treated cells can be isolated, and also tracked with fluorescence microscopy over long periods of time. This approach is useful for observing the protein downregulation and phenotypic responses of cells to gene regulation over time.

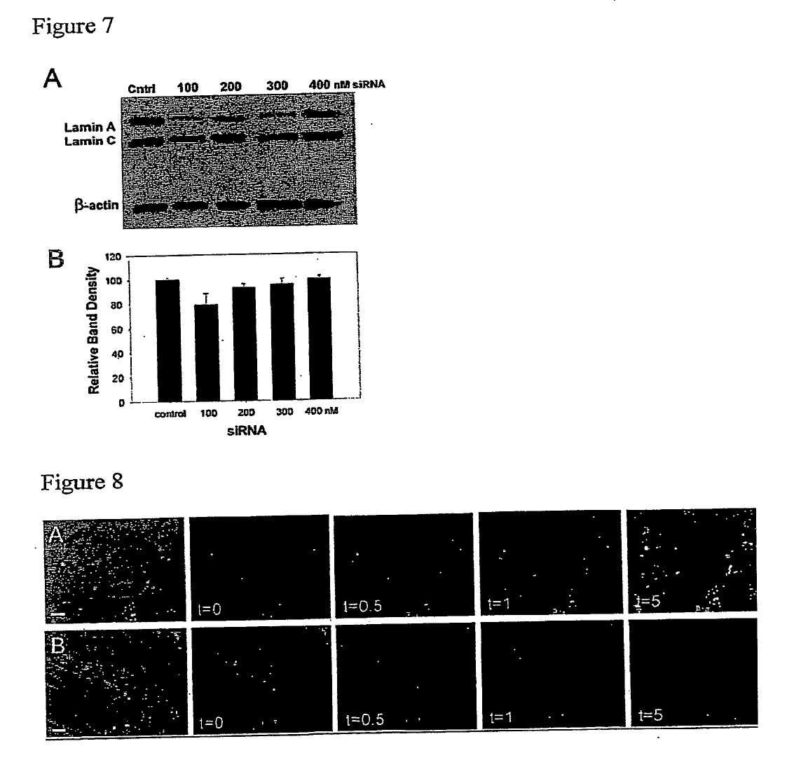

We note that attempts to improve silencing by simply using higher concentrations of siRNA do not improve knockdown but may actually negatively regulate RNAi-mediated gene silencing (FIG. 7) (40, 41). In addition, excesses of either siRNA or cationic liposome has been shown to induce increased cytotoxicity, interferon response (42) and “off-target” effects (43).

### Example 2

### Optimizing the Correlation Between QD Fluorescence and Gene Silencing

### Materials and Methods

QD and siRNA synthesis, transfection, and Western blotting were performed as described in Example 1.

### Results

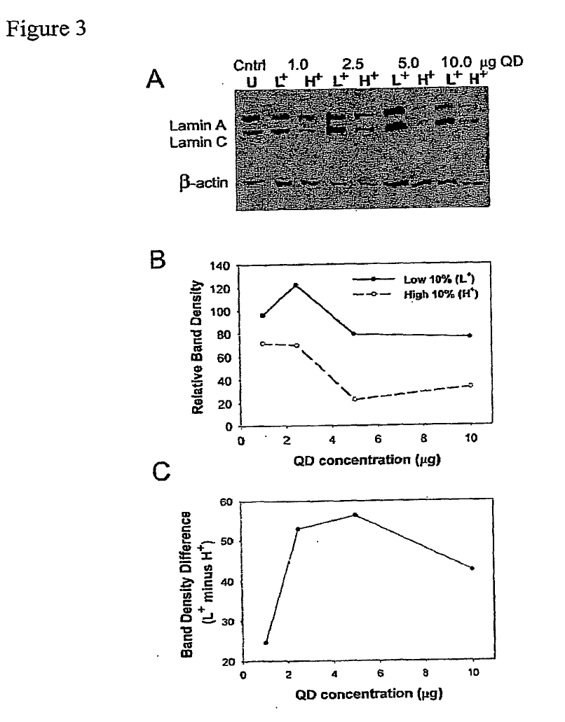

To optimize the QD/siRNA correlative effect, we varied the ratio of QD to lipofection reagent with a fixed dose of 100 nM siRNA. Specifically, we co-complexed Lmna siRNA with QD:lipofection reagent ratios of 1:5, 1:2, 1:1 or 2:1 (corresponding to 1, 2.5, 5 or 10 μg QD) and sorted the high 10% and low 10% of the cell fluorescence distributions as before. We found that optimal fluorescence and gene silencing correlation for the least amount of QD occurs at a 1:1 QD:lipofection reagent mass ratio (5 μg QD), as assayed by Western blot (FIG. 3A-C). Without wishing to be bound by any theory, we hypothesize that this optimum results from the limited surface area of the cationic liposome delivery agent (˜1 μm²) that is shared by the siRNA and QDs during the complexing process. Using too few QDs fails to provide fluorescence that is detectable over background, whereas excess QDs occupy sites on the liposome that would otherwise be available to siRNA. In support of this theory, we found that saturating the liposome with QDs (100:1 ratio) prior to transfection abolished correlation between cellular fluorescence and gene silencing; both high- and low-populations exhibited little to no knockdown (data not shown).

### Example 3

### Multiplexed Assay Allows Simultaneous Monitoring and Sorting of Cells Treated with Different siRNAs

### Materials and Methods

QD and siRNA synthesis, transfection, and Western blotting were performed as described in Example 1.

### Results

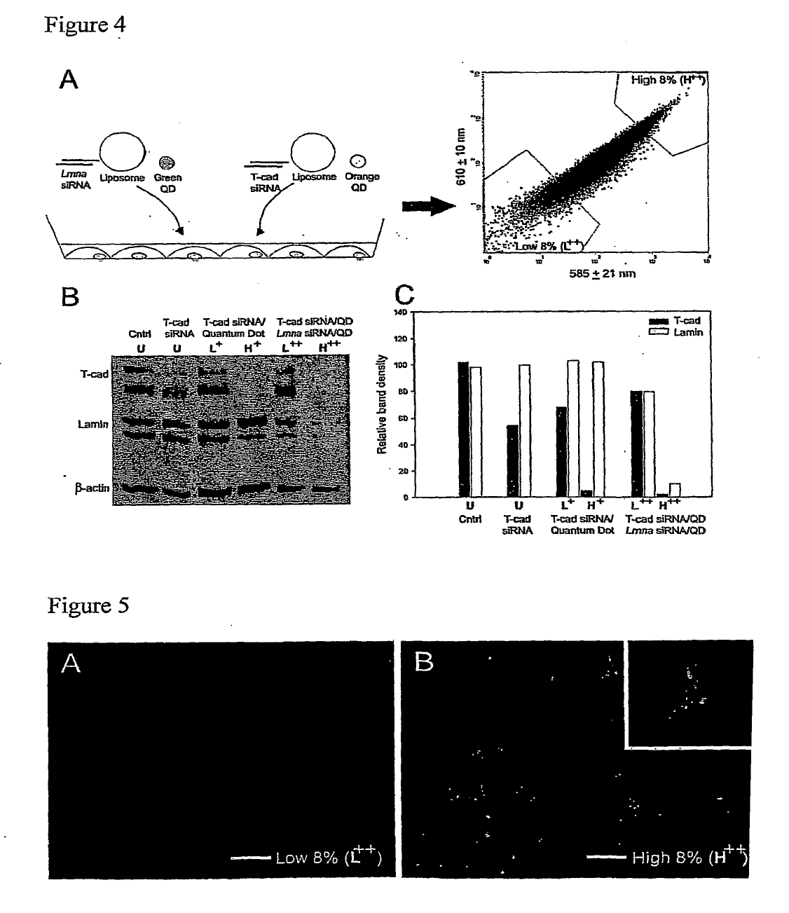

QDs exhibit an extensive range of size- and composition-dependent optical properties, making them highly advantageous for multiplexing (i.e. monitoring and sorting cells that have been treated simultaneously with different siRNA/QD complexes). As a demonstration of these capabilities, we complexed cationic liposomes with either green (em 560 nm) QDs and Lmna siRNA or orange (em 600 nm) QDs and siRNA targeting T-cadherin (T-cad). Cells were exposed simultaneously to both complexes and flow cytometry was used to quantify orange fluorescence (600±10 nm) versus green fluorescence (560±20 nm) (FIG. 4A). Cells exhibiting dual-color fluorescence were gated for low 8% and high 8% fluorescence and collected. Western blots probing lamin A/C and T-cad protein confirm specificity of QD/siRNA complexing (FIG. 4B and FIG. 4C), while fluorescence microscopy validates gating accuracy and demonstrates multi-color tracking capabilities (FIG. 5). Unsorted cells transfected with T-cad siRNA alone expressed a 45% down-regulation in protein expression quantified by Western blot band densitometry. In contrast, co-delivery of QDs with T-cad siRNA and subsequent sorting enabled separation of the least efficiently transfected cell subpopulation (30% protein knockdown) from a highly transfected population (95% knockdown). In the highest 8% of the dual color, dual siRNA co-transfected cell population, highly effective silencing of both Lmna gene (96% knockdown) and T-cad gene (98% knockdown) was achieved. Given the wide spectrum of QD color possibilities, this method promises to be useful for tracking and sorting multiple siRNA-mediated knockdowns within one cell population.

### Example 4

### Isolation of a Homogeneously Silenced Population of Fibroblasts Reveals a Role for T-Cadherin in Cell-Cell Communication Between Hepatocytes and Non-Parenchymal Cells

### Materials and Methods

QD and siRNA synthesis and transfection were performed as described in Example 1.

Hepatocyte/Fibroblast Co-Cultures

Hepatocytes were isolated from 2-3 month old adult female Lewis rats (Charles River Laboratories) and purified as described previously (34,35). Fresh, isolated hepatocytes were seeded at a density of 2.5×10⁵ cells per well, in 17-mm wells adsorbed with 0.13 mg/mL Collagen-I. Cultures were maintained at 37° C., 5% CO₂ in hepatocyte medium consisting of DMEM with high glucose, 10% fetal bovine serum, 0.5 U/mL insulin, 7 ng/mL glucagons, 7.5 μg/mL hydrocortisone, 10 U/mL penicillin, and 10 μg/mL streptomycin. Twenty-four hours after hepatocyte seeding, fibroblasts from transfection experiments were co-cultivated at a previously optimized 1:1 hepatocyte:fibroblast ratio in fibroblast medium (36). Medium from hepatocyte/fibroblast co-cultures was collected and replaced with hepatocyte medium every 24 hours until completion of the experiment.

Hepatocellular Function Assays

Hepatocyte/fibroblast co-cultures were assayed for albumin production and cytochrome P450 enzymatic activity, prototypic indicators of hepatocellular function (37, 38). Albumin content in spent media samples was measured using an enzyme linked immunosorbent assay (ELISA) with horseradish peroxidase detection (35). Cytochrome P450 (CYP1A1) enzymatic activity was measured by quantifying the amount of resorufin produced from the CYP-mediated cleavage of ethoxyresorufin O-deethylase EROD) (39). Specifically, EROD was incubated with cell cultures for 30 min, media was collected, and resorufin fluorescence quantified at 571/585 nm excitation/emission. Error bars represent standard error of the mean (n=3). Statistical significance was determined using one-way ANOVA (analysis of variance).

### Results

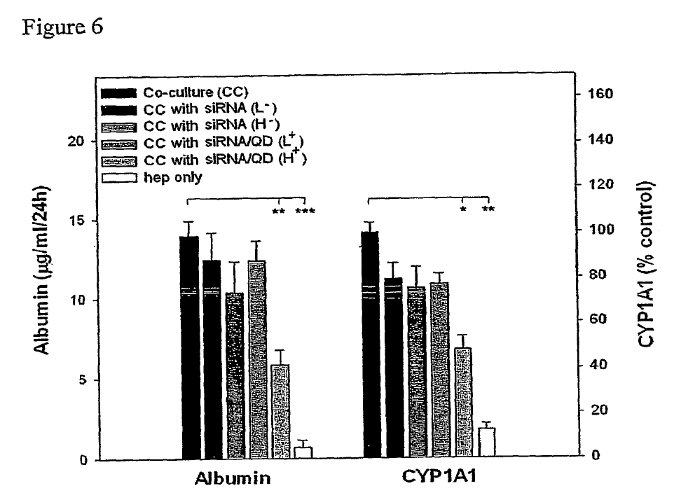

The utility of RNAi as a functional genomics tool is predicated upon associating gene silencing with downstream phenotypic observations. Yet non-uniform gene silencing may obscure bulk measurements (protein, mRNA) commonly used to validate gene knockdown and obscure genotype/phenotype correlations. We compared the downstream effects of non-uniform and homogenous gene silencing to specifically examine the stabilizing effect of non-parenchymal cells (3T3 fibroblasts) on hepatocellular function in vitro (36). Recently, several cadherins from hepatocyte-fibroblast junctions were identified as potential mediators of liver-specific function in vitro (37). Based on this finding, we transfected fibroblasts with T-cad siRNA or T-cad siRNA/QD complexes, sorted each population according to high or low cellular fluorescence, and co-cultivated the populations with hepatocytes. Markers of liver-specific function, albumin synthesis and cytochroine P450 1A1 (CYP1A1) activity, were measured in hepatocyte/3T3 co-cultures (FIG. 6). Compared to control co-cultures, significant downregulation in hepatocellular function (2-fold) was observed exclusively in the cultures that had been treated with T-cad siRNA/QD complexes and sorted for high cellular fluorescence. These studies implicate a role for fibroblast T-cadherin protein expression in modulating hepatocellular function in vitro, an interpretation revealed only once a homogenously-silenced population of fibroblasts was obtained.

### Example 5

### QDs Demonstrate Superior Photostability and Brightness Relative to Fluorescent Dyes for siRNA Tracking

### Materials and Methods

QD and siRNA synthesis and transfection were performed as described in Example 1.

### Results

Cells were transfected with 20 μg QD (em 566 nm) or 100 nM Lamin A/C siRNA modified with fluorescein on the 5′ end of the sense strand. As shown in FIG. 8A, QDs fluoresce brightly under continuous mercury lamp exposure over several minutes, while the fluoroscein attached to the siRNA bleaches under continuous excitation and is no longer detectable after t=5 minutes (FIG. 8B).

### Example 6

### Uptake and Silencing Activity of QD/siRNA Conjugates

### Materials and Methods

Quantum dots (Amino PEG ITK 705, Quantum Dot Corporation) were dissolved in 150 mM NaCl, 50 mM Sodium Phosphate, pH 7.2. 300 μg of cross-linker (SPDP, Pierce or SMCC, Sigma) was added per 500 pmol of nanoparticles and allowed to react for 1 hour. After filtering on a NAP5 gravity column to remove excess cross-linker, QDs were added to a 10 fold excess (5 mmol) of thiolated siRNA (first reduced with 0.1 M DTT and then filtered on a NAP5 column). The siRNA used was designed against destabilized enhanced GFP (“EGFP”, Clontech), and thiolated on the 5′ end of the sense strand. After reaction overnight at 4° C., particles were washed twice with PBS, twice with 5×SSC (1.5 M NaCl, 0.15 M Sodium Citrate, pH 7.2), and twice with PBS, using three Amicon-4 (100 kDa cutoff) spin filters. QDs were added to lipofectamine 2000 (1 μL per well of a 24 well plate) and allowed to complex for 20 minutes in serum-free media. QD/lipofectamine complexes were then added to GFP+HeLa cells (20-40% confluent in a 24 well plate). Media was changed to 10% FBS at 24 hours. Cells were trypsinized and flow cytometry performed at 48 hours to assess GFP and QD signal. Percent knockdown was assessed by comparing with control cells treated with lipofectamine alone.

### Results

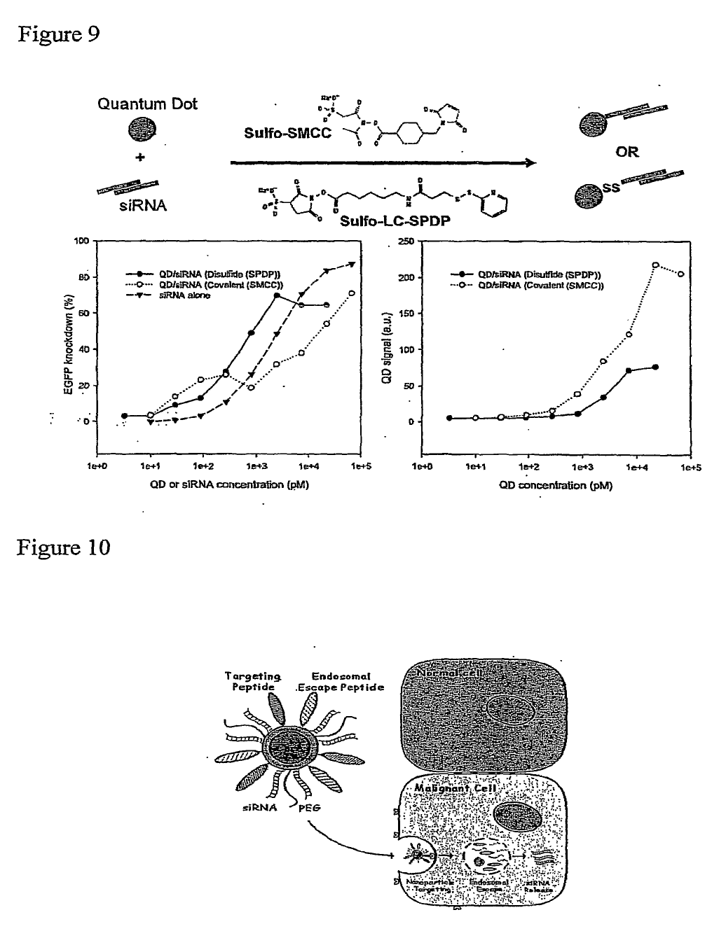

QDs and siRNA targeted to EGFP were conjugated to one another using either sulfo-SMCC or sulfo-LC-SPDP (depicted in the upper portion of FIG. 9) to produce QD/siRNA conjugates. The latter reagent provided conjugation via a disulfide bond. Complexes containing either Lipofectamine and siRNA or Lipofectamine and QD/siRNA conjugates were formed as described above. HeLa cells expressing EGFP were treated with Lipofectamine/siRNA complexes or with either of the two Lipofectamine/QD/siRNA complexes at a range of different QD concentrations. EGFP fluorescence was measured as an indication of EGFP expression. Fluorescence signal from the QD/siRNA complexes was gathered. As shown in FIG. 9 (left panel) both QD/siRNA conjugates resulted in efficient silencing of EGFP, with the disulfide-linked conjugate displaying a greater silencing effect under these conditions although the QD/siRNA conjugates produced using SMCC were taken up in higher amounts by the cells as shown in FIG. 9 (right panel). The apparently greater efficacy of the disulfide-linked conjugates may reflect release of the siRNA from the QD inside the cells.

### Example 7

### Targeted Delivery of QDs to Cells

### Materials and Methods

Quantum dots were conjugated to various peptides using sulfo-SMCC and the procedure described in Example 6 above. Briefly, 300 μg of cross-linker was added to 500 pmol of quantum dots. After 1 hour at room temperature, QDs were filtered on a NAP5 column and added to various thiolated peptides: KAREC (SEQ ID NO: 3), INF7, F3, F3+INF7 (equal molar ratio). KAREC denotes a 5 amino acid peptide, which is used as a non-internalizing control. 100 nM concentration of QDs were added to HeLa cells in media with 10% FBS. “No QDs” indicates no quantum dots were added to the cells and represents the background signal. “No peptide” indicates no peptide was added to the QDs after the cross-linker was added and particles filtered. Four hours later, cells were washed, trypsinized and flow cytometry was performed.

### Results

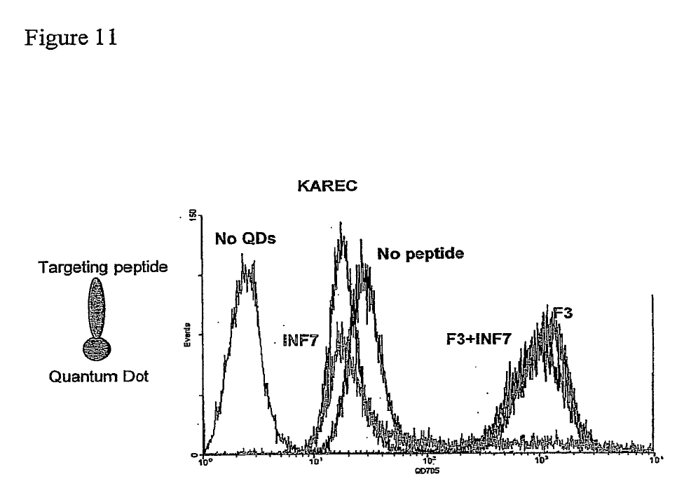

F3 (CAKVKDEPQRRSARLSAKPAPPKPEPKPKKAPAKK, SEQ ID: 4) is a 34 amino acid basic peptide that binds to nucleolin, a protein that is present at higher levels on the surface of dividing than non-dividing cells. INF7 (GLFEAIEGFI ENGWEGMI DGWYGC, SEQ ID NO: 5) is a peptide derived from the N-terminus of the influenza HA-2 domain that enhances endosome escape. QD/peptide conjugates were prepared in which QDs were conjugated either with F3, with INF7, with both F3 and INF7, or with the random control peptide (KAREC). Cells were treated with each preparation and analyzed for QD internalization by flow cytometry. As shown in FIG. 11 (right panel), the greatest internalization was achieved using QDs conjugated with either F3 alone or F3 and INF7, thereby demonstrating the ability of F3 to enhance QD uptake. In another experiment, QDs are conjugated with an siRNA, and the ability of the various conjugates to silence expression of a target gene is assessed.

### Example 8

### Optimization of Targeted QD/siRNA Conjugates

### Materials and Methods

Materials

Quantum dots with emission maxima of 655 nm or 705 nm and modified with PEG and amino groups were obtained from Quantum Dot Corporation (ITK amino). QD concentrations were measured by optical absorbance at 595 nm, using extinction coefficients provided by the supplier. Cross-linkers used were sulfo-LC-SPDP (sulfosuccinimidyl 6-(3′-[2-pyridyldithio]-propionamido)hexanoate) (Pierce) and sulfo-SMCC (sulfosuccinimidyl-4-(N-maleimidomethyl)cyclohexane-1-carboxylate) (Sigma). Synthetic RNA duplexes directed against the EGFP mRNA were synthesized, with the sense strand modified to contain a 5′ thiol group (Dharmacon) (Sense: 5′-Th-(CH₂)₆-GGC UAC GUC CAG GAG CGC ACC; Antisense: 5′-UGC GCU CCU GGA CGU AGC CUU). The F3 peptide was synthesized with an animohexanoic acid (Ahx) spacer and cysteine residue added for conjugation (Final sequence: C[Ahx]AKVK DEPQR RSARL SAKPA PPKPE PKPKK APAKK). A FITC-labeled F3 peptide was also synthesized, along with KAREC (Lys-Ala-Arg-Glu-Cys), a five amino acid control peptide. All peptides were synthesized by N-(9-fluorenylmethoxycarbonyl)-L-amino acid chemistry with a solid-phase synthesizer and purified by HPLC. The composition of the peptides was confirmed by MS.

Conjugation of Peptides and Nucleic Acid to QDs

Amino-modified QDs were conjugated to thiol-containing siRNA and peptides using sulfo-LC-SPDP and sulfo-SMCC cross-linkers. QDs were resuspended in 50 mM sodium phosphate, 150 mM sodium chloride, pH 7.2, using Amicon Ultra-4 (100 kDa cutoff) filters. Cross-linker (1000-fold excess) was added to QDs and allowed to react for 1 hour. Samples were filtered on a NAP-5 gravity column (to remove excess cross-linker) into similar buffer supplemented with 10 mM EDTA. siRNA was treated with 0.1 M DTT for one hour and filtered on a NAP-5 column into EDTA-containing buffer. Peptides were typically used from lyophilized powder. Peptide and/or siRNA was added to filtered QDs and allowed to react overnight at 4° C. Using three Amicon filters, product was filtered twice with Dulbecco's phosphate buffered saline (PBS), twice with a high salt buffer (1.0 M sodium chloride, 100 mM sodium citrate, pH 7.2), and twice again with PBS. High salt washes were performed to remove electrostatically bound siRNA and peptide, which was not removed with PBS washes alone.

For siRNA-QDs, a 10-fold excess of siRNA was typically used for both cross-linkers. In the case of sulfo-LC-SPDP, the amount of conjugated siRNA was assayed using gel electrophoresis (20% TBE gel, Invitrogen), staining with SYBR Gold (Invitrogen). To confirm that similar amounts of siRNA (approximately 2 per QD) were conjugated to QDs using sulfo-SMCC, particles were stained with SYBR Gold and measured with a fluorimeter (SpectraMax Gemini XS, Molecular Devices).

For F3/siRNA-QDs and KAREC/siRNA-QDs, a molar ratio of 15:70:1 (siRNA:peptide:QDs) was found to be optimum, though a variety of ratios were attempted (FIG. 4A). These conditions yielded approximately 20 F3 peptides and 1 siRNA duplex per particle.

Cell Culture

Internalization and knockdown experiments were performed using a HeLa cell line stably transfected with 1 h destabilized EGFP (courtesy of Phillip Sharp, MIT). Growth media was Dulbecco's modified Eagle's medium (DMEM) containing 4.5 g/L glucose and supplemented with 10% FBS, 100 units/mL penicillin, 100 ug/mL streptomycin, and 292 ug/mL L-glutamine. Cells were passaged into 24-well plates and used at 50-80% confluency for internalization experiments and 20-40% confluency for knockdown experiments.

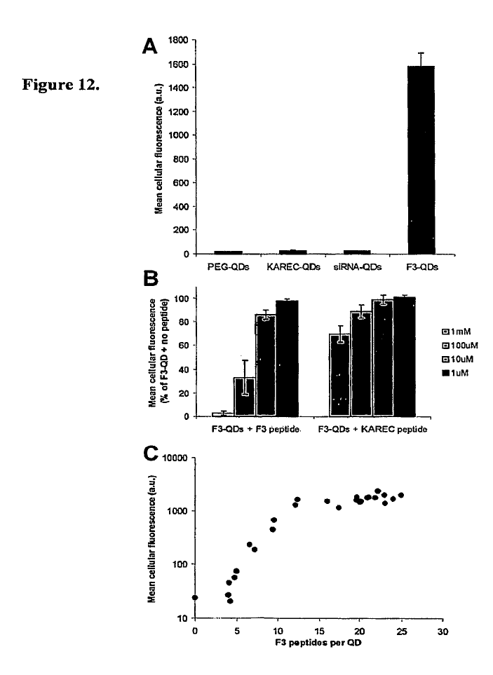

For internalization experiments (FIG. 12), QDs were added to cell monolayers in media without serum at a final concentration of 50 nM. After four hours, cells were washed with media, treated with trypsin (0.25%) and EDTA, and resuspended in 1% BSA (in PBS) for flow cytometry (BD FACSort, FL1 for EGFP signal and FL3 for QD signal). Fluorescence data on 10,000 cells were collected for each sample and the geometric mean of intensity was reported.

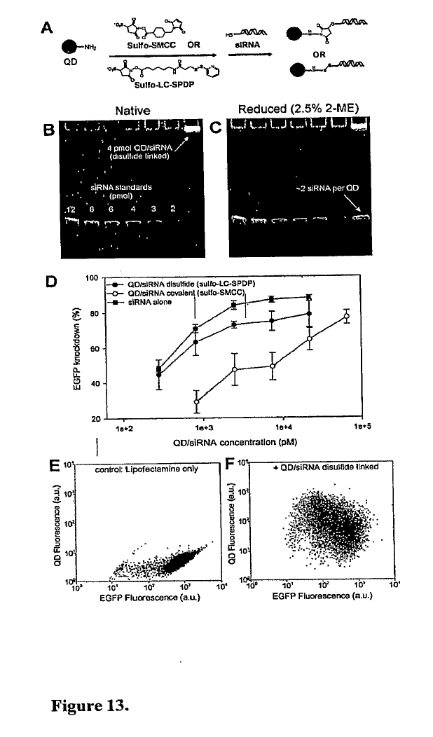

For knockdown experiments (FIG. 13), siRNA-QDs (in 50 μL serum/antibiotic-free media) were added to Lipofectamine 2000 (1 μL in 50 μL media, Invitrogen) and allowed to complex for 20 minutes. Cell media was changed to 400 μL of serum/antibiotic-free per well, and QD solutions (100 μL) were added dropwise. Complete media was added 12-18 hours later. Forty-eight hours after the QD were added, cells were trypsinized and assayed for fluorescence by flow cytometry.

To assess EGFP knockdown, 50 nM or 10 nM concentrations of F3/siRNA-QDs or KAREC/siRNA-QDs were added to cell monolayers (20-40% confluent) in media with serum/antibiotics. Four hours later, cells were washed with similar media. Some samples were then treated with 1 μL of Lipofectamine per well (added dropwise in 100 μL media) either immediately after washing or after a 90 minute incubation at 37° C. (to allow membrane recycling). For all samples, media was changed to complete DMEM with serum/antibiotics ˜16 hours after the addition of QDs, and assayed by flow cytometry 48 hours from the start of the experiment. For imaging, cells were initially seeded on glass-bottom dishes (Mat-Tek) and observed 48 hours after the addition of QDs using a 60× oil immersion objective. Images were captured with a SPOT camera mounted on a Nikon TE200 inverted epifluorescence microscope.

### Results

Taking a modular approach, particle internalization and siRNA attachment were investigated separately before these functions were combined in a single particle. First, peptides were conjugated to QDs to improve tumor cell uptake. Addition of as-purchased PEGlyated QDs to HeLa cell monolayers led to minimal cell uptake, as quantified with flow cytometry (FIG. 12A). Conjugation of siRNA or a control pentapeptide (KAREC) did not increase QD internalization, but addition of F3 peptide to the QDs improved the uptake significantly (two orders of magnitude). To confirm the specificity of F3 uptake, free F3 peptide was added to cells along with 50 nM F3-QDs (FIG. 12B). Dose-dependent inhibition of uptake was observed with F3 peptide concentrations from 1 μM to 1 mM. Inhibition of uptake by free KAREC peptide was minimal by comparison. The large excess of free peptide required for inhibition may be due to multiple copies of the F3 peptide on each QD and improved receptor binding as a result of multivalency.

To quantify the number of peptides added per particle, FITC-labeled F3 peptide was synthesized and attached to QDs using a cleavable cross-linker (sulfo-LC-SPDP). After filtering to remove unreacted peptide, 2-mercaptoethanol (2-ME) was added to reduce the disulfide bond between peptide and QD. Using a 100 kDa cutoff filter, F3-FITC peptide was separated from the QDs and quantified by fluorescence. Several reactions were performed with various amounts of FITC-F3 and siRNA as reactants. For each formulation, the cellular uptake was quantified by flow cytometry and F3 number measured (FIG. 12C, each point indicates a separate formulation). The results suggest that up to ˜25 F3 peptides can be added per QD. Attachment of a small number of peptides (0-5) did not lead to significant uptake (less than 10% of maximum). Uptake increased with peptide number, but began to saturate around 15 copies per QD.

The use of cleavable (sulfo-LC-SPDP) or non-cleavable (sulfo-SMCC) cross-linkers for the attachment of F3 peptide did not significantly affect cell uptake. The choice of cross-linker, however, may affect the ability of the siRNA cargo to interact with RISC. The interior of the cell is a reducing environment, which would lead to cleavage of the disulfide bond generated by sulfo-LC-SPDP, freeing the siRNA. On the other hand, the amide bond produced by sulfo-SMCC is unaffected by reducing conditions (confirmed by treating the conjugates with 2.5% 2-ME for 30 min), leaving the intracellular QD/siRNA conjugate intact. We compared the efficiency of QD/siRNA conjugates prepared with both cross-linkers using an EGFP model system. Delivery of the conjugates to EGFP-labeled HeLa cells was performed by first complexing the particles with a cationic liposome transfection reagent (Lipofectamine 2000), to satisfy the functions of cell internalization and endosome escape, and knockdown efficiency was quantified by a reduction in EGFP fluorescence over controls (Lipofectamine only).

Using gel electrophoresis, the amount of siRNA conjugated per particle was quantified relative to double-stranded RNA standards. Particles conjugated using sulfo-LC-SPDP were first introduced under native (non-reduced) conditions (FIG. 13B). The absence of a siRNA band in the QD/siRNA lanes indicates that no siRNA is electrostatically bound to the particles. Exposing the particles to 2-ME for 30 minutes led to the appearance of a siRNA band in the SPDP lane, which can be quantified with RNA standards and ImageQuant software (FIG. 13C). Using this approach, approximately two siRNA duplexes were conjugated per QD under these conditions. Cellular fluorescence was quantified 48 hours after incubation with HeLa cells using flow cytometry. As hypothesized, the QD/siRNA formulation produced with the disulfide bond (using sulfo-LC-SPDP) led to greater EGFP knockdown (FIG. 3D).

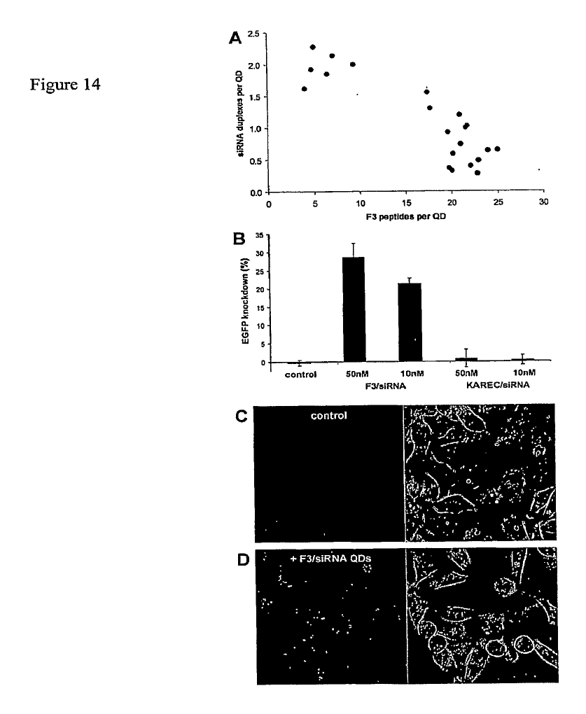

In addition to improved siRNA function, the use of a cleavable cross-linker allows the removal and quantification of both species after F3 peptide and siRNA co-attachment. The F3:siRNA reaction ratio was varied with the goal of generating a formulation capable of high cell uptake as well as the ability to carry a significant payload of siRNA. The results indicate a trade-off between one siRNA per particle with high uptake (>15 peptides) and two duplexes but low uptake (<10 peptides) (FIG. 14A). Negatively-charged siRNA may be electrostatically adsorbing to the surface of the aminated QDs, preventing the attachment of additional F3 peptides. Potentially, performing the reaction in high salt conditions, or in the presence of a surfactant, may allow higher loading. Since both high uptake efficiency and siRNA number are required for knockdown, particles with ˜20 F3s and a single siRNA duplex were further investigated.

When incubated with cells, these particles were shown to internalize significantly, but did not lead to reduction in EGFP fluorescence 48 hours later. Fluorescence microscopy revealed that the particles were intracellular, but they colocalized with an endosomal marker (LysoSensor, Molecular Probes). Addition of an endosome escape agent, therefore, was used to achieve knockdown. Specifically, after incubation of cells with F3/siRNA-QDs and washing, cationic liposomes were added for 12 hours. Although cationic liposomes and polymers are typically used to form complexes with nucleic acids or particles, thereby ferrying the payload inside cells, in this case the reagent led to endosomal escape of previously internalized QDs. Without wishing to be bound by any theory, the cationic liposomes may be internalized into new endosomes, which fuse with the endosomes carrying the QDs. As the pH of the vesicle is lowered by the cell, osmotic lysis leads to the release of both species into the cytoplasm. To assess the importance of the targeting ligand, particles carrying siRNA and a control peptide (KAREC) were used. These KAREC/siRNA particles were not internalized, and no EGFP knockdown was observed, despite endosome disruption. Additionally, a time lag of 90 minutes between washing the cells free of QDs and cationic liposome addition did not lead to significant reduction in efficiency, indicating that endosomal degradation of the siRNA is not an issue on this time scale.

In addition to cationic liposomes, some chemotherapeutics, such as chloroquine have been shown to result in endosomal escape (Won et al., 2005, Science, 309:121). While an endosome escape step could be a realistic part of a treatment regimen, there is also potential that this function could be built into each particle. Addition of a fusogenic peptide to the QD surface, for example, may further improve delivery of the multifunctional particles described (Plank et al., 1994, J. Biol. Chem., 269:12918).

Decorating the surface of a fluorescent quantum dot with both a targeting ligand and siRNA duplex requires a tradeoff in the number of each species but can be used to generate a conjugate capable of knockdown in vitro. We found that multiple copies of the F3 targeting peptide were required for QD uptake, but that siRNA cargo could be co-attached without affecting the function of the peptide. Disulfide (sulfo-LC-SPDP) and covalent (sulfo-SMCC) cross-linkers were investigated for the attachment of siRNA to the particle, with the disulfide bond showing greater silencing efficiency. Finally, after delivery to cells and release from their endosomal entrapment, F3/siRNA-QDs led to knockdown of EGFP signal. By designing the siRNA sequence against a therapeutic target (e.g. an oncogene) instead of EGFP, this technology may be adapted to treat and image metastatic cancer. The technology explored in this study could be readily adapted to other nanoparticle platforms, such as iron oxide or gold cores, which allow image contrast on magnetic resonance or x-ray imaging respectively and may therefore mitigate concerns over QD cytotoxicity and the limited tissue penetration of light. QDs, however, remain an attractive tool for in vitro and animal testing, where fluorescence is the most accessible and common imaging modality.

### EQUIVALENTS

Those skilled in the art will recognize, or be able to ascertain using no more than routine experimentation, many equivalents to the specific embodiments of the invention, described herein. The scope of the present invention is not intended to be limited to the above Description, but rather is as set forth in the appended claims.

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nanoparticle type, property, or material composition; any RNA type; any transfection reagent, etc.) can be excluded from any one or more claims, for any reason, whether or not related to the existence of prior art.

### REFERENCES

1. Fire, A., Xu, S., Montgomery, M. K., Kostas, S. A., Driver, S. E. and Mello, C. C. (1998) Potent and specific genetic interference by double-stranded RNA in Caenorhabditis elegans. Nature, 391, 806-811.
2. Kamath, R. S., Fraser, A. G., Dong, Y., Poulin, G., Durbin, R., Gotta, M., Kanapin, A., Le Bot, N., Moreno, S., Sohrmann, M. et al. (2003) Systematic functional analysis of the Caenorhabditis elegans genome using RNAi. Nature, 421, 231-237.
3. Maeda, I., Kohara, Y., Yamamoto, M. and Sugimoto, A. (2001) Large-scale analysis of gene function in Caenorhabditis elegans by high-throughput RNAi. Curr Biol, 11, 171-176.
4. Boutros, M., Kiger, A. A., Armknecht, S., Kerr, K., Hild, M., Koch, B., Haas, S. A., Consortium, H. F., Paro, R. and Perrimon, N. (2004) Genome-wide RNAi analysis of growth and viability in Drosophila cells. Science, 303, 832-835.
5. Zheng, L., Liu, J., Batalov, S., Zhou, D., Orth, A., Ding, S. and Schultz, P. G. (2004) An approach to genomewide screens of expressed small interfering RNAs in mammalian cells. Proc Natl Acad Sci USA, 101, 135-140.
6. Novina, C. D. and Sharp, P. A. (2004) The RNAi revolution. Nature, 430, 161-164.
7. Chi, J. T., Chang, H. Y., Wang, N. N., Chang, D. S., Dunphy, N. and Brown, P. O. (2003) Genomewide view of gene silencing by small interfering RNAs. Proc Natl Acad Sci USA, 100, 6343-6346.
8. Semizarov, D., Frost, L., Sarthy, A., Kroeger, P., Halbert, D. N. and Fesik, S. W. (2003) Specificity of short interfering RNA determined through gene expression signatures. Proc Natl Acad Sci USA, 100, 6347-6352.
9. Raab, R. M. and Stephanopoulos, G. (2004) Dynamics of gene silencing by RNA interference. Biotechnol Bioeng, 88, 121-132.
10. Huppi, K., Martin, S. E. and Caplen, N. J. (2005) Defining and assaying RNAi in mammalian cells. Mol Cell, 17, 1-10.
11. Spagnou, S., Miller, A. D. and Keller, M. (2004) Lipidic carriers of siRNA: differences in the formulation, cellular uptake, and delivery with plasmid DNA. Biochemistry, 43, 13348-13356.
12. Oberdoerffer, P., Kanellopoulou, C., Heissmeyer, V., Paeper, C., Borowski, C., Aifantis, I., Rao, A. and Rajewsky, K. (2005) Efficiency of RNA interference in the mouse hematopoietic system varies between cell types and developmental stages. Mol Cell Biol, 25, 3896-3905.
13. McManus, M. T. and Sharp, P. A. (2002) Gene silencing in mammals by small interfering RNAs. Nat Rev Genet, 3, 737-747.
14. Muratovska, A. and Eccles, M. R. (2004) Conjugate for efficient delivery of short interfering RNA (siRNA) into mammalian cells. FEBS Lett, 558, 63-68.
15. Lorenz, C., Hadwiger, P., John, M., Vornlocher, H. P. and Unverzagt, C. (2004) Steroid and lipid conjugates of siRNAs to enhance cellular uptake and gene silencing in liver cells. Bioorg Med Chem Lett, 14, 4975-4977.
16. Schiffelers, R. M., Ansari, A., Xu, J., Zhou, Q., Tang, Q., Storm, G., Molema, G., Lu, P. Y., Scaria, P. V. and Woodle, M. C. (2004) Cancer siRNA therapy by tumor selective delivery with ligand-targeted sterically stabilized nanoparticle. Nucleic Acids Res, 32, e149.
17. Itaka, K., Kanayama, N., Nishiyama, N., Jang, W. D., Yamasaki, Y., Nakamura, K., Kawaguchi, H. and Kataoka, K. (2004) Supramolecular nanocarrier of siRNA from PEG-based block catiomer carrying diamine side chain with distinctive pKa directed to enhance intracellular gene silencing. J Am Chem Soc, 126, 13612-13613.
18. Manoharan, M. (2004) RNA interference and chemically modified small interfering RNAs. Curr Opin Chem Biol, 8, 570-579.
19. Chiu, Y. L., Ali, A., Chu, C. Y., Cao, H. and Rana, T. M. (2004) Visualizing a correlation between siRNA localization, cellular uptake, and RNAi in living cells. Chem Biol, 11, 1165-1175.
20. Kumar, R., Conklin, D. S. and Mittal, V. (2003) High-throughput selection of effective RNAi probes for gene silencing. Genome Res, 13, 2333-2340.
21. Hemann, M. T., Fridman, J. S., Zilfou, J. T., Hernando, E., Paddison, P. J., Cordon-Cardo, C., Hannon, G. J. and Lowe, S. W. (2003) An epi-allelic series of p53 hypomorphs created by stable RNAi produces distinct tumor phenotypes in vivo. Nat Genet, 33, 396-400.
22. Wu, X., Liu, H., Liu, J., Haley, K. N., Treadway, J. A., Larson, J. P., Ge, N., Peale, F. and Bruchez, M. P. (2003) Immunofluorescent labeling of cancer marker Her2 and other cellular targets with semiconductor quantum dots. Nat Biotechnol, 21, 41-46.
23. Dahan, M., Levi, S., Luccardini, C., Rostaing, P., Riveau, B. and Triller, A. (2003) Diffusion dynamics of glycine receptors revealed by single-quantum dot tracking. Science, 302, 442-445.
24. Tsien, R. Y. (1998) The green fluorescent protein. Annu Rev Biochem, 67, 509-544.
25. Derfus, A. M., Chan, W. C. and Bhatia, S. (2004) Probing the cytotoxicity of semiconductor quantum dots. Nano Letters, 4, 11-18.
26. Mattheakis, L. C., Dias, J. M., Choi, Y. J., Gong, J., Bruchez, M. P., Liu, J. and Wang, E. (2004) Optical coding of mammalian cells using semiconductor quantum dots. Anal Biochem, 327, 200-208.
27. Gao, X., Cui, Y., Levenson, R. M., Chung, L. W. and Nie, S. (2004) In vivo cancer targeting and imaging with semiconductor quantum dots. Nat Biotechnol, 22, 969-976.
28. Derfus, A. M., Chan, W. C. W. and Bhatia, S. N. (2004) Intracellular Delivery of Quantum Dots for Live Cell Labeling and Organelle Tracking. Advanced Materials, 16, 961-966.
29. Chan, W. C. and Nie, S. (1998) Quantum dot bioconjugates for ultrasensitive nonisotopic detection. Science, 281, 2016-2018.
30. Hines, M. A. and Guyot-Sionnest, P. (1996) Synthesis and characterization of strongly luminescing ZnS-Capped CdSe nanocrystals. Journal of Physical Chemistry, 100, 468-471.
31. Dabbousi, B. O., RodriguezViejo, S., Mikulec, F. V., Heine, J. R., Mattoussi, H., Ober, R., Jensen, K. F. and Bawendi, M. G. (1997) (CdSe)ZnS core-shell quantum dots: Synthesis and characterization of a size series of highly luminescent nanocrystallites. Journal of Physical Chemistry B, 101, 9463-9475.
32. Rheinwald, J. G. and Green, H. (1975) Serial cultivation of strains of human epidermal keratinocytes: the formation of keratinizing colonies from single cells. Cell, 6, 331-343.
33. Ranscht, B. and Dours-Zimmermann, M. T. (1991) T-cadherin, a novel cadherin cell adhesion molecule in the nervous system lacks the conserved cytoplasmic region. Neuron, 7, 391-402.
34. Seglen, P. O. (1976) Preparation of isolated rat liver cells. Methods Cell Biol, 13, 29-83.
35. Dunn, J. C., Tompkins, R. G. and Yarmush, M. L. (1991) Long-term in vitro function of adult hepatocytes in a collagen sandwich configuration. Biotechnol Prog, 7, 237-245.
36. Bhatia, S. N., Balis, U. J., Yarmush, M. L. and Toner, M. (1999) Effect of cell-cell interactions in preservation of cellular phenotype: cocultivation of hepatocytes and nonparenchymal cells. Faseb J, 13, 1883-1900.
37. Khetani, S., Szulgit, G., Rio, J. D., Barlow, C. and Bhatia, S. N. (2004) Exploring Interactions Between Hepatocytes and Nonparenchymal Cells Using Gene Expression Profiling. Hepatology., 40, 545-554.
38. Allen, J. W., Johnson, R. S. and Bhatia, S. N. (2005) Hypoxic inhibition of 3-methylcholanthrene-induced CYP1A1 expression is independent of HIF-1alpha. Toxicol Lett, 155, 151-159.
39. Behnia, K., Bhatia, S., Jastromb, N., Balis, U., Sullivan, S., Yarmush, M. and Toner, M. (2000) Xenobiotic metabolism by cultured primary porcine hepatocytes. Tissue Eng, 6, 467-479.
40. Hong, J., Qian, Z., Shen, S., Min, T., Tan, C., Xu, J., Zhao, Y. and Huang, W. (2005) High doses of siRNAs induce eri-1 and adar-1 gene expression and reduce the efficiency of RNA interference in the mouse. Biochem J, 390, 675-679.
41. Kennedy, S., Wang, D. and Ruvkun, G. (2004) A conserved siRNA-degrading RNase negatively regulates RNA interference in C. elegans. Nature, 427, 645-649.
42. Sledz, C. A., Holko, M., de Veer, M. J., Silverman, R. H. and Williams, B. R. (2003) Activation of the interferon system by short-interfering RNAs. Nat Cell Biol, 5, 834-839.
43. Jackson, A. L., Bartz, S. R., Schelter, J., Kobayashi, S. V., Burchard, J., Mao, M., Li, B., Cavet, G. and Linsley, P. S. (2003) Expression profiling reveals off-target gene regulation by RNAi. Nat Biotechnol, 21, 635-637.
44. Hirko, A., Tang, F. and Hughes, J. A. (2003) Cationic lipid vectors for plasmid DNA delivery. Curr Med Chem, 10, 1185-1193.
45. Ge Q, Filip L, Bai A, Nguyen T, Eisen H N, and Chen J. (2004) Inhibition of influenza virus production in virus-infected mice by RNA interference. Proc Natl Acad Sci USA, 101 (23):8676-81.
46. Radhakrishnan S K, Layden T J, Gartel A L. (2004) RNA interference as a new strategy against viral hepatitis. Virology 323 (2):173-81.
47. Hu W Y, Bushman F D, Siva A C. (2004) RNA interference against retroviruses. Virus Res. 102 (1):59-64.
48. Kohrer C, Xie L, Kellerer S, Varshney U, RajBhandary U L. (2001) Import of amber and ochre suppressor tRNAs into mammalian cells: a general approach to site-specific insertion of amino acid analogues into proteins. Proc Natl Acad Sci USA 98 (25):14310-5.
49. Zamore, P. D., et al. (2000) RNAi: double-stranded RNA directs the ATP-dependent cleavage of mRNA at 21 to 23 nucleotide intervals.
50. Elbashir, S. M., et al. (2001) RNA interference is mediated by 21- and 22-nucleotide RNAs. Genes Dev. 15: 188-200.
51. Yuan, B, et al., (2004) siRNA Selection Server: an automated siRNA oligonucleotide prediction server. Nucl. Acids. Res. 32:W130-W134.
52. Santoyo J, Vaquerizas J M, Dopazo J. (2005) Highly specific and accurate selection of siRNAs for high-throughput functional assays. Bioinformatics. 21 (8):1376-82. Epub 2004 Dec. 10.
53. Reynolds, A., et al. (2004) Rational siRNA design for RNA interference. Nat. Biotechnol. 22: 326-330.
54. Dykxhhorn, D, M., Novina, C. D., and Sharp, P. A. (2003) Killing the messenger: short RNAs that silence gene expression. Nature Reviews Molecular Cell Biology. 4: 457-467.
55. Hannon, G. J. and Rossi, J. J. (2004) Unlocking the potential of the human genome with RNA interference. Nature. 431: 3761-378.
56. Meister, G. and Tuschl, T. (2004) Mechanisms of gene silencing by double-stranded RNA. Nature. 431
57. Kohrer C, Sullivan E L, RajBhandary UL. (2004) Nucleic Acids Res. Complete set of orthogonal 21st aminoacyl-tRNA synthetase-amber, ochre and opal suppressor tRNA pairs: concomitant suppression of three different termination codons in an mRNA in mammalian cells. 32 (21):6200-11.
58. Zheng, J, et al., (2004) Highly Fluorescent, Water-Soluble, Size-Tunable Gold Quantum Dots, Phys. Rev. Lett., 93 (7).
59. Alivisatos A P, Gu W, Larabell C. (2005) Quantum dots as cellular probes. Annu Rev Biomed Eng. 7:55-76.
60. Hotz C Z (2005) Applications of quantum dots in biology: an overview. Methods Mol. Biol. 303:1-17.
61. Bagwe, R. P., Yang, C., Hilliard, L. R. and Tan, W. (2004) Optimization of dye-doped silica nanoparticles prepared using a reverse microemulsion method. Langmuir 20, 8336.
62. Van Blaaderen, A. and Vrij, A. (1992) Synthesis and characterization of colloidal dispersions of fluorescent, monodisperse silica spheres. Langmuir 8, 2921.
63. Lin, Y., et al., Well-ordered mesoporous silica nanoparticles as cell markers, J. Am. Chem. Soc., 17, 4570-4573 (2005).
64. Zhao, X., et al., (2004) Adv. Mat., 16 (2), 173-176.
65. Wang, L., et al., (2005) Nano Letters, Vol. 5 (1), 37-43.
66. Ow, H., et al., (2005) Nano Letters, Vol. 5 (1), 113-117.
67. Gao, X., et al. In vivo molecular and cellular imaging with quantum dots. Curr. Op. in Biotechnol., 16: 63-72.
68. Kelly, K. L., Coronado, E., Zhao, L. L. and Schatz, G. C. The optical properties of metal nanoparticles: the influence of size, shape and dielectric environment. J. Phys. Chem. B. 107, 668 (2003).
69. Schultz, S., Smith, D. R., Mock, J. J. and Schultz, D. A (2000) Single-target molecule detection with nonbleaching multicolor optical immunolables. Proc. Natl. Acad. Sci. USA 97, 996.
70. Schultz, D., (2003) Plasmon resonant particles for biological detection. Curr. Op. Biotechnol. 14:13-22.
71. Jackson, J. B. and Halas, N. J., (2004) Surface-enhanced Raman scattering on tunable plasmonic nanoparticle substrates Proc. Natl. Acad. Sci. USA 101 (52): 17930-17935
72. Link, S. and El-Sayed, M. A., (2003) Optical Properties and Ultrafast Dynamics of Metallic Nanocrystals. Annu. Rev. Phys. Chem. 54, 331-66.
73. Masala, O. and Seshadri, R. (2004) Synthesis Routes for Large Volumes of Nanoparticles. Annu. Rev. Mater. Res. 34, 41-81.
74. Beaureparie, E., et al., (2004) Functionalized Fluorescent Oxide Nanoparticles: Artificial Toxins for Sodium Channel Targeting and Imaging at the Single-Molecule Level, Nano Letters 4, 2079.
75. Wind, R. A., et al. (2000) An integrated confocal and magnetic resonance microscope for cellular research. J. Magn. Reson. 147: 371-77.
76. Wietzorrek, J., et al. (1999) A new multiparameter flow cytometer: optical and electrical cell analysis in combination with video microscopy in flow. Cytometry 35: 291-301.
77. Kamentsky L A. (2001) Laser scanning cytometry. Methods Cell Biol., 63:51-87.
78. Safarik, I, and Safarikova, M., (1999) Use of magnetic techniques for isolation of cells. J. Chromatogr. B Biomed. Sci. Appl., 722, 33-53.
79. Wang, M M, et al, (2005) Microfluidic sorting of mammalian cells by optical force switching. Nat. Biotechnol. 23 (1): 83-7.
80. Yao, B., et al. (2004) A microfluidic device based on gravity and electric force driving for flow cytometry and fluorescence activated cell sorting. Lab Chip. 4 (6): 603-7.
81. Chiou, P. Y., et al., (2005) Massively parallel manipulation of single cells and microparticles using optical images. Nature. 436: 370-72.
82. Singh, M, et al., (2002) Biotechnol Adv. 20 (5-6):341-59.
83. Somiari S, Glasspool-Malone J, Drabick J J, Gilbert R A, Heller R, Jaroszeski M J, Malone R W. (2002) Theory and in vivo application of electroporative gene delivery. Mol. Ther. 2 (3):178-8.
84. Nikoloff, Jac A., ed. (1995) Animal Cell Electroporation and Electrofusion Protocols, Methods in Molecular Biology, Volume 48, Humana Press, Totowa, N.J.
85. Boas, U. and Heegaard, P M, (2004) Chem Soc Rev., 33 (1):43-63.
86. Elouhabi, A. and Ruysschaert, J-M. (2005) Formation and intracellular trafficking of lipoplexes and polyplexes. Mol. Ther. 11 (3): 336-347.
87. Heyes, J., et al., (2005) Cationic lipid saturation influences intracellular delivery of encapsulated nucleic acids. J. Cont. Rel. 107: 276-287.
88. Thomas M, Lu J J, Ge Q, Zhang C, Chen J, Klibanov A M. (2005) Full deacylation of polyethylenimine dramatically boosts its gene delivery efficiency and specificity to mouse lung. Proc Natl Acad Sci USA; 102 (16):5679-84.
89. Gemeinhart, R. A., Luo, D. & Saltzman. Cellular Fate of a Modular DNA Delivery System Mediated by Silica Nanoparticles. American Chemical Society and American Institute of Chemical Engineers (2005).
90. Oishi, M., Nagasaki, Y., Itaka, K., Nishiyama, N. & Kataoka, K. Lactosylated poly(ethylene glycol)-siRNA conjugate through acid-labile beta-thiopropionate linkage to construct pH-sensitive polyion complex micelles achieving enhanced gene silencing in hepatoma cells. J Am Chem Soc 127, 1624-5 (2005).
91. Wagner, E., Plank, C., Zatloukal, K., Cotten, M. & Bimstiel, M. L. Influenza virus hemagglutinin HA-2 N-terminal fusogenic peptides augment gene transfer by transferrin-polylysine-DNA complexes: toward a synthetic virus-like gene-transfer vehicle. Proc Natl Acad Sci USA 89, 7934-8 (1992).
92. Porkka, K., Laakkonen, P., Hoffman, J. A., Bemasconi, M. & Ruoslahti, E. A fragment of the HMGN2 protein homes to the nuclei of tumor cells and tumor endothelial cells in vivo. Proc Natl Acad Sci USA 99, 7444-9 (2002).
93. Christian, S. et al. Nucleolin expressed at the cell surface is a marker of endothelial cells in angiogenic blood vessels. J Cell Biol 163, 871-8 (2003).
94. Mastrobattista, E. et al. Functional characterization of an endosome-disruptive peptide and its application in cytosolic delivery of immunoliposome-entrapped proteins. J Biol Chem 277, 27135-43 (2002).
95. Wadia, J. S., Stan, R. V. & Dowdy, S. F. Transducible TAT-HA fusogenic peptide enhances escape of TAT-fusion proteins after lipid raft macropinocytosis. Nat Med 10, 310-5 (2004).
96. Won, J. et al. A magnetic nanoprobe technology for detecting molecular interactions in live cells. Science 309, 121-5 (2005).

1. An isolated composition comprising an optically or magnetically detectable nanoparticle and an RNAi agent.

2. The composition of claim 1, wherein the nanoparticle is physically associated with the RNAi agent.

3. The composition of claim 1, wherein the nanoparticle is not conjugated to the RNAi agent.

4. The composition of claim 1, wherein the nanoparticle is conjugated to the RNAi agent.

5. The composition of claim 1, wherein the RNAi agent is an siRNA.

6. The composition of claim 1, wherein the nanoparticle comprises a quantum dot.

7. The composition of claim 1, wherein the nanoparticle comprises a plasmon resonant particle.

8. The composition of claim 1, wherein the nanoparticle comprises a fluorescent or luminescent moiety.

9. The composition of claim 1, further comprising a transfection reagent comprising one or more materials selected from the group consisting of: cationic lipids, non-cationic lipids, cationic polymers, non-cationic polymers, dendrimers, polysaccharides, dextran, and translocation peptides.

10. The composition of claim 1, further comprising a transfection reagent, wherein the nanoparticle, the RNAi agent, and the transfection reagent form a complex.

11. (canceled)

12. The composition of claim 1, wherein the composition comprises (i) at least first and second optically or magnetically detectable nanoparticles having distinguishable optical or magnetic properties, and (ii) at least first and second RNAi agents, wherein the first optically or magnetically detectable nanoparticle is physically associated with the first RNA agent and the second optically or magnetically detectable nanoparticle is physically associated with the second RNAi agent.

13. The composition of claim 1, further comprising (i) at least one additional optically or magnetically detectable nanoparticle having optical or magnetic properties distinguishable from those of the first and second nanoparticles, and (ii) at least one additional RNAi agent, wherein each additional RNAi agent is associated with a nanoparticle having optical or magnetic properties distinguishable from those of the nanoparticles that are physically associated with the other RNAi agents.

14. (canceled)

15. The composition of claim 1, wherein the optically or magnetically detectable nanoparticle, the RNAi agent, or both, has an endosomal escape agent attached thereto.

16. The composition of claim 1, wherein the optically or magnetically detectable nanoparticle, the RNAi agent, or both, has a cell targeting agent attached thereto.

17. The composition of claim 1, wherein the optically or magnetically detectable nanoparticle is attached to the RNAi agent by a cleavable linkage.

18-30. (canceled)

31. A kit comprising: an optically or magnetically detectable nanoparticle and an RNAi agent.

32-40. (canceled)

41. A method of monitoring delivery of a functional RNA to a cell comprising steps of:
(a) contacting the cell with an optically or magnetically detectable nanoparticle and a functional RNA; and
(b) analyzing the cell to detect the presence, absence, or amount of the nanoparticle in the cell, wherein presence of the nanoparticle in the cell is indicative of presence of the functional RNA in the cell.

42. (canceled)

43. The method of claim 41, wherein the amount of the nanoparticle in the cell is indicative of the amount of the functional RNA in the cell.

44. The method of claim 41, wherein the amount of the nanoparticle in the cell correlates with activity of the functional RNA in the cell.

45. The method of claim 41, wherein the functional RNA is an RNAi agent and the amount of the nanoparticle in the cell correlates with gene silencing activity of the RNAi agent in the cell.

46-48. (canceled)

49. The method of claim 41, wherein the step of analysing comprises performing FACS, imaging, or fluorescence microscopy.

50-69. (canceled)

70. A method of sorting cells comprising steps of:
(a) contacting cells with an optically or magnetically detectable nanoparticle and a functional RNA;
(b) analyzing the cells to detect the presence, absence, or amount of the nanoparticle in the cells; and
(c) identifying the cells as belonging to one of at least two populations based on the presence, absence, or amount of the nanoparticle in the cells.

71-73. (canceled)

74. The method of claim 70, further comprising: physically separating the cells into at least two populations based on the presence, absence, or amount of the nanoparticle in the cells.

75-82. (canceled)

83. A method of monitoring gene silencing in a cell comprising steps of:
(a) contacting the cell with an optically or magnetically detectable nanoparticle and an RNAi agent targeted to a gene; and
(b) analyzing the cell to detect the presence, absence, or amount of the nanoparticle in the cell, wherein presence of the nanoparticle in the cell is indicative of silencing of the gene by the RNAi agent.

84. The method of claim 83, wherein the amount of the nanoparticle in the cell is indicative of the degree of silencing of the gene.

85. (canceled)

86. The method of claim 83, wherein the amount of the nanoparticle in the cell is indicative of the amount of the RNAi agent in the cell.

87. The method of claim 83, wherein the RNAi agent is an siRNA and the amount of the nanoparticle in the cell correlates with gene silencing activity of the siRNA in the cell.

88-115. (canceled)
